(19) United States
(12) Patent Application Publication
(10) Pub. No.: US 2006/0014960 A1
(43) Pub. Date: Jan. 19, 2006

(54) Substituted pyridines or pyrimidines, method for their production, agents containing said substances and the use thereof as pesticides

(75) Inventors: Marion Beckmann, Wiesbaden (DE); Uwe Doller, Rodgau (DE); Gerhard Krautstrunk, Bad Vilbel (DE); Wolfgang Schaper, Diedorf (DE); Daniela Jans, Bad Homburg (DE); Waltraud Hempel, Liederbach (DE)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Bayer CropScience GmbH, Frankfurt (DE)

(21) Appl. No.: 10/525,819

(22) PCT Filed: Aug. 14, 2003
(86) PCT No.: PCT/EP03/09028

§ 371 Date: Feb. 25, 2005

(30) Foreign Application Priority Data
Aug. 30, 2002 (DE) 102 39 905.0

(51) Int. Cl. C07D 239/36; C07D 213/56

(52) U.S. Cl. 546/315; 544/335

(57) ABSTRACT

The invention relates to compounds of the formula (I) 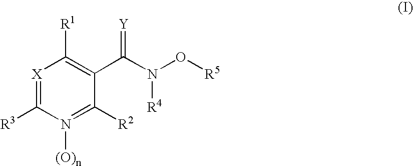 where the symbols and indices are defined as cited in the description. Said compounds are suitable for controlling pests.

The present invention relates to heterocyclic hydroxamic acid derivatives, to processes for their preparation, to compositions comprising them and to their use for controlling animal pests, in particular arthropods, such as insects and arachnids, and helminths, for example nematodes.

Owing to the enormous damage caused by insects, for example by feeding on useful plants, stored food, wood and textiles, or else by transferring diseases to man, domestic animals and useful plants, the use of insecticides or repellants remains indispensable. Insecticides are an important component of integrated pest control, and their contribution is decisive with respect to harvest yields and yield continuity all over the world.

Trifluoromethylpyri(mi)dinehydroxamic acid derivatives are known as pesticides from WO-A-01/09104. The general formula of the compounds described also encompasses pyri(mi)dine-N-acyl-hydroxamic acid derivatives. Pyri(mi)dine-N-aryl-acylhydroxamic acid derivatives and pyri(mi)dine-N-(methyl)benzoyl-hydroxamic acid derivatives are disclosed specifically.

However, since the ecological and economic demands made on modern insecticides are increasing continually, for example with respect to toxicity, selectivity, application rates, formation of residues and favorable manufacture, and there can furthermore be problems, for example with resistance, there is a constant need to develop novel insecticides which, at least in some areas, have advantages over those of the prior art.

It has been found that compounds of the formula (I), if appropriate also as salts, have a good activity spectrum against animal pests and at the same time good compatibility with plants and favorable toxicological properties with respect to mammals and aquatic animals.

Accordingly, the invention provides compounds of the formula (I) and salts thereof, 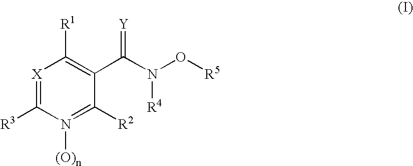
where the symbols and indices are as defined below:
X is ═CH— or ═N—;
Y is ═O or ═S;
n is 0 or 1;
R¹ is (C₁-C₆)-alkyl, (C₁-C₆)-haloalkyl, —S(halogen)₅ or halogen, where one or two CH₂ groups may be replaced by —O— or —S— or —N(C₁-C₆)-alkyl, with the proviso that heteroatoms may not be adjacent;
R², R³ independently of one another are hydrogen, (C₁-C₆)-alkyl, (C₁-C₆)-haloalkyl or halogen, where one or two CH₂ groups may be replaced by —O— or —S— or —N(C₁-C₆)-alkyl, with the proviso that heteroatoms may not be adjacent;
R⁴ is hydrogen, (C₁-C₆)-alkyl, (C₃-C₁₀)-alkenyl, (C₃-C₁₀)-alkynyl, (C₃-C₁₀)-cycloalkyl, (C₄-C₈)-cycloalkenyl, (C₈-C₁₀)-cycloalkynyl, (C₆-C₁₄)-aryl, (C₃-C₁₀)-heterocyclyl or R⁶, where the radicals mentioned may optionally be mono- or polysubstituted;
R⁵ is hydrogen, (C₁-C₁₀)-alkyl, (C₃-C₁₀)-alkenyl, (C₃-C₁₀)-alkynyl, (C₃-C₈)-cycloalkyl, (C₄-C₈)-cycloalkenyl, (C₈-C₁₀)-cycloalkynyl, (C₆-C₁₄)-aryl, (C₃-C₁₀)-heterocyclyl or R⁷, where the radicals mentioned may optionally be mono- or polysubstituted;
R⁶, R⁷ independently of one another are —C(W)R⁸, —C(W)OR⁸, —C(W)SR⁸, —C(W)NR⁸R₂, —C(W)NR⁸R⁸—NR⁸₂, —C(W)NR⁸—NR⁸[C(W) R⁸], —SO₂NR⁸₂, —SO₂OR⁸, —S(O)R⁸₁—S(O)₂R⁸, —PWR⁸₂ or —PW(OR⁸)₂;
W is ═O, ═S, ═NOR⁸ or ═NNR⁸₂;
the radicals R⁸ are identical or different and are hydrogen, (C₁-C₆)-alkyl, (C₂-C₆)-alkenyl, (C₂-C₆)-alkynyl, (C₃-C₈)-cycloalkyl, (C₄-C₈)-cycloalkenyl, (C₃-C₈)-cycloalkyl-(C₁-C₄)-alkyl, (C₄-C₈)-cycloalkenyl-(C₁-C₄)-alkyl, (C₃-C₈)-cycloalkyl-(C₂-C₄)-alkenyl, (C₄-C₈)-cycloalkenyl-(C₂-C₄)-alkenyl, (C₁-C₆)-alkyl-(C₃-C₈)-cycloalkyl, (C₂-C₆)-alkenyl-(C₃-C₈)-cycloalkyl, (C₂-C₆)-alkynyl-(C₃-C₈)-cycloalkyl, (C₁-C₆)-alkyl-(C₄-C₈)-cycloalkenyl, (C₂-C₆)-alkenyl-(C₄-C₈)-cycloalkenyl, (C₆-C₁₄)-aryl, (C₃-C₁₀)-heterocyclyl, where the radicals mentioned may optionally be mono- or polysubstituted and two radicals R⁸ together optionally form a ring system;
with the proviso that at least one of the radicals R⁴ or R⁵ has one of the meanings defined for R⁶ or R⁷ and that, if R⁵ is —C(═O)R^{a}, where R^{a} is (C₁-C₆)-alkyl or (C₆-C₁₄)-aryl and where the radicals mentioned may optionally be mono- or polysubstituted, R⁴ is hydrogen or optionally mono- or polysubstituted (C₃-C₁₀)-alkenyl, (C₃-C₁₀)-alkynyl, (C₃-C₁₀)-cycloalkyl, (C₄-C₈)-cycloalkenyl, (C₈-C₁₀)-cycloalkynyl or R⁶.

The symbols and indices in formula (I) are preferably as defined below:
X is preferably ═CH—;
Y is preferably ═O;
n is preferably 0;
R¹ is preferably (C₁-C₆)-haloalkyl or —SF₅, in particular (C₁-C₆)-alkyl which is mono- or polysubstituted by F and/or Cl, particularly preferably CF₃, CHF₂ or CF₂Cl, very particularly preferably CF₃;
R², R³ are preferably hydrogen, halogen, (C₁-C₆)-alkyl, (C₁-C₆)-alkoxy, NH(C₁-C₆)-alkyl, N(C₁-C₆)₂-alkyl, particularly preferably hydrogen;
R⁴ is preferably hydrogen, (C₁-C₆)-alkyl, (C₁-C₆)-alkyl which is mono- or polysubstituted by F and/or Cl, particularly preferably hydrogen or CH₃, or R⁶;
R⁵ is preferably (C₁-C₆)-alkyl, (C₃-C₆)-alkenyl, (C₃-C₆)-alkynyl, (C₃-C₈)-cycloalkyl, (C₆-C₁₄)-aryl or (C₃-C₁₀)-heterocyclyl having a total of one to three nitrogen, oxygen and/or sulfur ring atoms or very particularly preferably R⁷, where the radicals mentioned may optionally be mono- or polysubstituted;
R⁶ and R⁷ independently of one another are preferably —C(W)R⁸, —C(W)OR⁸, —SO₂OR⁸, —S(O)R⁸, —S(O)₂R⁸, —PWR⁸₂ or —PW(OR⁸)₂;
W is preferably ═O;
the radicals R⁸ are identical or different and are preferably hydrogen, (C₁-C₆)-alkyl, (C₂-C₆)-alkenyl, (C₂-C₆)-alkynyl, (C₃-C₈)-cycloalkyl, (C₆-C₁₄)-aryl, (C₃-C₁₀)-heterocyclyl having a total of one to three nitrogen, oxygen and/or sulfur ring atoms, where the radicals mentioned may optionally be mono- or polysubstituted.

Particularly preferred are those compounds of the formula (I) where the symbols and indices are as defined below:
X is ═CH—;
Y is ═O;
n is O;
R¹ is —CF₃,
R² and R³ are hydrogen,
R⁴ is hydrogen, —C(W)R⁸, —S(O)R⁸ or —S(O)₂R⁸;
R⁵ is (C₁-C₆)-alkyl, (C₃-C₆)-alkenyl, (C₃-C₆)-alkynyl, (C₃-C₈)-cycloalkyl, (C₆-C₁₄)-aryl, (C₃-C₁₀)-heterocyclyl having a total of one to three nitogren, oxygen and/or sulfur ring atoms, —C(W)R⁸, —S(O)R⁸ or —S(O)₂R⁸;
the radicals R⁸ are identical or different and are preferably hydrogen, (C₁-C₆)-alkyl, (C₂-C₆)-alkenyl, (C₂-C₆)-alkynyl, (C₃-C₈)-cycloalkyl, (C₆-C₁₄)-aryl, (C₃-C₁₀)-heterocyclyl having a total of one to three nitrogen, oxygen and/or sulfur ring atoms, where the radicals mentioned may optionally be mono- or polysubstituted.

Very particular preference is given to those compounds of the formula (I) where the symbols and indices are as defined below:
X is ═CH—;
Y is ═O;
n is O;
R¹ is —CF₃;
R² and R³ are hydrogen;
R⁴ is —C(W)R⁸ and in particular hydrogen;
R⁵ is —C(W)R⁸, and
the radicals R⁸ are identical or different and are hydrogen, (C₁-C₆)-alkyl, (C₂-C₆)-alkenyl, (C₂-C₆)-alkynyl, (C₃-C₈)-cycloalkyl, (C₆-C₁₄)-aryl, (C₃-C₁₀)-heterocyclyl having a total of one to three nitrogen, oxygen and/or sulfur ring atoms, where the radicals mentioned may optionally be mono- or polysubstituted.

Preferred substituents on the radicals R⁴, R⁵ and R⁸ are groups R⁹ which are as defined below:
the radicals R⁹ are identical or different and are R¹⁰, or two radicals R⁹ together with the atoms to which they are attached form a three- to eight-membered saturated or unsaturated ring system which is optionally substituted by one or more radicals R¹⁰ and which optionally contains heteroatoms, preferably O, N, S, SO and/or SO₂;
the radicals R¹⁰ are identical or different and are R⁸, R¹¹, —C(W)R⁸, —C(W)OR⁸, —C(W)SR⁸, —C(W)NR⁸₂, —OC(W)R⁸, —OC(O)OR⁸, —OC(W)SR⁸, —OC(W)NR⁸₂, —SC(W)R⁸, —SC(W)OR⁸, —SC(W)SR⁸, —SC(W)NR⁸₂, —NR⁸C(W)R⁸, —N[C(M)R⁸]₂, —NR⁸C(W)OR⁸, —NR⁸C(W)SR⁸, —C(W)NR⁸—NR⁸₂, —C(W)NR⁸—NR⁸[C(W)R⁸], —NR⁸—C(W)NR⁸₂, —NR⁸—NR⁸C(W)R⁸, —NR⁸—N[C(W)R⁸]₂, —N[(CW)R⁸]—NR⁸₂, —NR⁸[(CW)NR⁸₂], —NR⁸(C═NR⁸)R⁸, —NR⁸(C═NR⁸)NR⁸₂, —O—NR⁸₂, —O—NR⁸(CW)R⁸, —SO₂NR⁸₂, —NR⁸SO₂R⁸, —SO₂OR⁸, —OSO₂R⁸, —OR⁸, —NR⁸₂, —SR⁸, —SiR⁸₃, —PR⁸₂, —P(W)R⁸₂, —SOR⁸—SO₂R⁸, —PWR⁸₂ or —PW(OR⁸)₂;
   or two radicals R¹⁰ together are (W), (═N—R⁸), (═CR₂′), (═CHR⁸), or (═CH₂);
W and R⁸ are as defined above;
the radicals R¹¹ are identical or different and are halogen, cyano, nitro, hydroxyl, thio, amino, formyl, (C₁-C₆)-alkanoyl, (C₁-C₆)-alkoxy, (C₃-C₆)-alkenyloxy, (C₃-C₆)-alkynyloxy, (C₁-C₆)-haloalkyloxy, (C₃-C₆)-haloalkenyloxy, (C₃-C₆)-haloalkynyloxy, (C₃-C₈)-cycloalkoxy, (C₄-C₈)-cycloalkenyloxy, (C₃-C₈)-halocycloalkoxy, (C₄-C₈)-halocycloalkenyloxy, (C₃-C₈)-cycloalkyl-(C₁-C₄)-alkoxy, (C₄-C₈)-cycloalkenyl-(C₁-C₄)-alkoxy, (C₃-C₈)-cycloalkyl-(C₂-C₄)-alkenyloxy, (C₄-C₈)-cycloalkenyl-(C₂-C₄)-alkenyloxy, (C₁-C₆)-alkyl-(C₃-C₈)-cycloalkoxy, (C₂-C₆)-alkenyl-(C₃-C₈)-cycloalkoxy, (C₂-C₆)-alkynyl-(C₃-C₈)-cycloalkoxy, (C₁-C₆)-alkyl-(C₄-C₈)-cycloalkenyloxy, (C₂-C₆)-alkenyl-(C₄-C₈)-cycloalkenyloxy, (C₁-C₄)-alkoxy-(C₁-C₆)-alkoxy, (C₁-C₄)-alkoxy-(C₃-C₆)-alkenyloxy, carbamoyl, (C₁-C₆)-mono- or dialkylcarbamoyl, (C₁-C₆)-mono- or dihaloalkylcarbamoyl, (C₃-C₈)-mono- or dicycloalkylcarbamoyl, (C₁-C₆)-alkoxycarbonyl, (C₃-C₈)-cycloalkoxycarbonyl, (C₁-C₆)-alkanoyloxy, (C₃-C₈)-cycloalkanoyloxy, (C₁-C₆)-haloalkoxycarbonyl, (C₁-C₆)-haloalkanoyloxy, (C₁-C₆)-alkanamido, (C₁-C₆)-haloalkanamido, (C₂-C₆)-alkenamido, (C₃-C₈)-cycloalkanamido, (C₃-C₈)-cycloalkyl-(C₁-C₄)-alkanamido, (C₁-C₆)-alkylthio, (C₃-C₆)-alkenylthio, (C₃-C₆)-alkynylthio, (C₁-C₆)-haloalkylthio, (C₃-C₆)-haloalkenylthio, (C₃-C₆)-haloalkynylthio, (C₃-C₈)-cycloalkylthio, (C₄-C₈)-cycloalkenylthio, (C₃-C₈)-halocycloalkthio, (C₄-C₈)-halocycloalkenylthio, (C₃-C₈)-cycloalkyl-(C₁-C₄)-alkylthio, (C₄-C₈)-cycloalkenyl-(C₁-C₄)-alkylthio, (C₃-C₈)-cycloalkyl-(C₃-C₄)-alkenylthio, (C₄-C₈)-cycloalkenyl-(C₃-C₄)-alkenylthio, (C₁-C₆)-alkyl-(C₃-C₈)-cycloalkylthio, (C₂-C₆)-alkenyl-(C₃-C₈)-cycloalkylthio, (C₂-C₆)-alkynyl-(C₃-C₈)-cycloalkylthio, (C₁-C₆)-alkyl-(C₄-C₈)-cycloalkenylthio, (C₂-C₆)-alkenyl-(C₄-C₈)-cycloalkenylthio, (C₁-C₆)-alkylsulfinyl, (C₃-C₆)-alkenylsulfinyl, (C₃-C₆)-alkynylsulfinyl, (C₁-C₆)-haloalkylsulfinyl, (C₃-C₆)-haloalkenylsulfinyl, (C₃-C₆)-haloalkynylsulfinyl, (C₃-C₈)-cycloalkylsulfinyl, (C₄-C₈)-cycloalkenylsulfinyl, (C₃-C₈)-halocycloalkylsulfinyl, (C₄-C₈)-halocycloalkenylsulfinyl, (C₃-C₈)-cycloalkyl-(C₁-C₄)-alkylsulfinyl, (C₄-C₈)-cycloalkenyl-(C₁-C₄)-alkylsulfinyl, (C₃-C₈)-cycloalkyl-(C₃-C₄)-alkenylsulfinyl, (C₄-C₈)-cycloalkenyl-(C₃-C₄)-alkenylsulfinyl, (C₁-C₆)-alkyl-(C₃-C₈)-cycloalkylsulfinyl, (C₂-C₆)-alkenyl-(C₃-C₈)-cycloalkylsulfinyl, (C₂-C₆)-alkynyl-(C₃-C₈)-cycloalkylsulfinyl, (C₁-C₆)-alkyl-(C₄-C₈)-cycloalkenylsulfinyl, (C₂-C₆)-alkenyl-(C₄-C₈)-cycloalkenylsulfinyl, (C₁-C₆)-alkylsulfonyl, (C₃-C₆)-alkenylsulfonyl, (C₃-C₆)-alkynylsulfonyl, (C₁-C₆)-haloalkylsulfonyl, (C₃-C₆)-haloalkenylsulfonyl, (C₃-C₆)-haloalkynylsulfonyl, (C₃-C₈)-cycloalkylsulfonyl, (C₄-C₈)-cycloalkenylsulfonyl, (C₃-C₈)-halocycloalkylsulfonyl, (C₄-C₈)-halocycloalkenylsulfonyl, (C₃-C₈)-cycloalkyl-(C₁-C₄)-alkylsulfonyl, (C₄-C₈)-cycloalkenyl-(C₁-C₄)-alkylsulfonyl, (C₃-C₈)-cycloalkyl-(C₃-C₄)-alkenylsulfonyl, (C₄-C₈)-cycloalkenyl-(C₃-C₄)-alkenylsulfonyl, (C₁-C₆)-alkyl-(C₃-C₈)-cycloalkylsulfonyl, (C₂-C₆)-alkenyl-(C₃-C₈)-cycloalkyl-sulfonyl, (C₂-C₆)-alkynyl-(C₃-C₈)-cycloalkylsulfonyl, (C₁-C₆)-alkyl-(C₄-C₈)-cycloalkenylsulfonyl, (C₂-C₆)-alkenyl-(C₄-C₈)-cycloalkenylsulfonyl, (C₁-C₆)-dialkylamino, (C₁-C₆)-alkylamino, (C₃-C₆)-alkenylamino, (C₃-C₆)-alkynylamino, (C₂-C₆)-haloalkylamino, (C₃-C₆)-haloalkenylamino, (C₃-C₆)-haloalkynylamino, (C₃-C₈)-cycloalkylamino, (C₄-C₈)-cycloalkenylamino, (C₃-C₈)-halocycloalkamino, (C₄-C₈)-halocycloalkenylamino, (C₃-C₈)-cycloalkyl-(C₁-C₄)-alkylamino, (C₄-C₈)-cycloalkenyl-(C₁-C₄)-alkylamino, (C₃-C₈)-cycloalkyl-(C₃-C₄)-alkenylamino, (C₄-C₈)-cycloalkenyl-(C₃-C₄)-alkenylamino, (C₁-C₆)-alkyl-(C₃-C₈)-cycloalkylamino, (C₂-C₆)-alkenyl-(C₃-C₈)-cycloalkylamino, (C₂-C₆)-alkynyl-(C₃-C₈)-cycloalkylamino, (C₁-C₆)-alkyl-(C₄-C₈)-cycloalkenylamino, (C₂-C₆)-alkenyl-(C₄-C₈)-cycloalkenylamino, (C₁-C₆)-trialkylsilyl, aryl, aryloxy, arylthio, arylamino, aryl-(C₁-C₄)-alkoxy, aryl-(C₃-C₄)-alkenyloxy, aryl-(C₁-C₄)-alkylthio, aryl-(C₂-C₄)-alkenylthio, aryl-(C₁-C₄)-alkylamino, aryl-(C₃-C₄)-alkenylamino, aryl-(C₁-C₆)-dialkylsilyl, diaryl-(C₁-C₆)-alkylsilyl, triarylsilyl and 5- or 6-membered heterocyclyl, where the cyclic moiety of the 14 last-mentioned radicals is optionally substituted by one or more radicals from the group consisting of halogen, cyano, nitro, amino, hydroxyl, thio, (C₁-C₄)-alkyl, (C₁-C₄)-haloalkyl, (C₃-C₈)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylamino, (C₁-C₄)-haloalkylamino, formyl and (C₁-C₄)-alkanoyl.
the radicals R¹¹ are preferably identical or different and are halogen, cyano, nitro, (C₁-C₆)-alkanoyl, (C₁-C₆)-alkoxy, (C₁-C₆)-haloalkyloxy, (C₃-C₈)-cycloalkoxy, (C₃-C₈)-cycloalkyl-(C₁-C₄)-alkoxy, (C₁-C₆)-mono- or -dialkylcarbamoyl, (C₁-C₆)-alkoxycarbonyl, (C₁-C₆)-haloalkoxycarbonyl, (C₁-C₆)-alkylthio, (C₁-C₆)-haloalkylthio, (C₃-C₈)-cycloalkylthio, (C₁-C₆)-alkylsulfinyl, (C₁-C₆)-haloalkylsulfinyl, (C₃-C₈)-cycloalkylsulfinyl, (C₁-C₆)-alkylsulfonyl, (C₁-C₆)-haloalkylsulfonyl, (C₃-C₈)-cycloalkyl-sulfonyl, (C₁-C₆)-dialkylamino, (C₁-C₆)-alkylamino, (C₃-C₈)-cycloalkyl-amino, (C₁-C₆)-trialkylsilyl, aryl, aryloxy, arylthio, aryl-(C₁-C₄)-alkyl, arylamino, aryl-(C₁-C₄)-alkoxy, where the cyclic moiety of the six last-mentioned radicals is optionally substituted by one or more radicals from the group consisting of halogen, nitro, (C₁-C₄)-alkyl, (C₁-C₄)-haloalkyl, (C₁-C₄)-alkoxy and (C₁-C₄)-haloalkoxy.

The term “halogen” embraces fluorine, chlorine, bromine and iodine. Chlorine or fluorine are preferred.

The term “—S(halogen)₅” embraces the groups —SI₅, —SBr₅, —SCl₅ and in particular —SF₅.

The term “(C₁-C₆)-alkyl” is to be understood as meaning a straight-chain or branched hydrocarbon radical having one to six carbon atoms, such as, for example, the methyl, ethyl, propyl, isopropyl, 1-butyl, 2-butyl, 2-methylpropyl, tert-butyl, 1-pentyl, 2-methylbutyl, 1,1-dimethylpropyl or 1-hexyl radical. Correspondingly, alkyl radicals having a wider range of carbon atoms are to be understood as meaning straight-chain or branched saturated hydrocarbon radicals comprising a number of carbon atoms which corresponds to this stated range.

“(C₁-C₆)-haloalkyl” is to be understood as meaning an alkyl group mentioned under the term “(C₁-C₆)-alkyl” in which one or more hydrogen atoms are replaced by the same number of identical or different halogen atoms, preferably chlorine or fluorine, such as the trifluoromethyl, the 1-fluoroethyl, the 2,2,2-trifluorethyl, the chloromethyl, fluoromethyl, the difluoromethyl and the 1,1,2,2-tetrafluoroethyl groups.

“(C₁-C₆)-alkoxy” is to be understood as meaning an alkoxy group whose hydrocarbon radical has the meaning given under the term “(C₁-C₆)-alkyl”. Alkoxy groups having a wider range of carbon atoms are to be understood likewise.

The terms “alkenyl” and “alkynyl” with a prefix in which the number of carbon atoms is stated are straight-chain or branched hydrocarbon radicals having a number of carbon atoms which corresponds to this stated range and comprising at least one multiple bond which may be in any position of the unsaturated radical in question. Accordingly, “(C₂-C₄)-alkenyl” is, for example, the vinyl, allyl, 2-methyl-2-propenyl or 2-butenyl group; “(C₂-C₆)-alkenyl” is the radicals mentioned above and also, for example, the pentenyl, 2-methylpentenyl or the hexenyl group. “(C₂-C₄)-alkynyl” is, for example, the ethynyl, propargyl, 2-methyl-2-propynyl or 2-butynyl group.

“(C₂-C₆)-alkynyl” is to be understood as meaning the radicals mentioned above and also, for example, the 2-pentynyl or the 2-hexynyl group, and “(C₂-C₁₀)-alkynyl” is to be understood as meaning the radicals mentioned above and also, for example, the 2-octynyl or the 2-decynyl group.

“(C₃-C₈)-cycloalkyl” is monocyclic alkyl radicals, such as the cyclopropyl, cyclobutyl, cyclopentyl, cyclohexyl, cycloheptyl or cyclooctyl radical, and bicyclic alkyl radicals, such as the norbornyl radical.

The term “(C₃-C₈)-cycloalkyl-(C₁-C₄)-alkyl” is to be understood as meaning, for example, the cyclopropylmethyl, cyclopentylmethyl, cyclohexylmethyl, cyclohexyl-ethyl and cyclohexylbutyl radical, and the term “(C₁-C₆)-alkyl-(C₃-C₈)-cycloalkyl” is to be understood as meaning, for example, the 1-methylcyclopropyl, 1-methylcyclo-pentyl, 1-methylcyclohexyl, 3-hexylcyclobutyl and the 4-tert-butylcyclohexyl radical.

“(C₁-C₄)-alkoxy-(C₁-C₆)-alkyloxy” is an alkoxy group as defined above which is substituted by a further alkoxy group, such as, for example, 1-ethoxyethoxy.

“(C₃-C₈)-cycloalkoxy” or “(C₃-C₈)-cycloalkylthio” is to be understood as meaning one of the (C₃-C₈)-cycloalkyl radicals listed above which is attached via an oxygen or sulfur atom.

“(C₃-C₈)-cycloalkyl-(C₁-C₆)-alkoxy” is, for example, the cyclopropylmethoxy, cyclobutylmethoxy, cyclopentylmethoxy, cyclohexylmethoxy, cyclohexylethoxy or the cyclohexylbutoxy group.

The term “(C₁-C₄)-alkyl-(C₃-C₈)-cycloalkoxy” denotes, for example, the methyl-cyclopropyloxy, methylcyclobutyloxy or the butylcyclohexyloxy group.

“(C₁-C₆)-alkylthio” is an alkylthio group whose hydrocarbon radical has the meaning given under the term “(C₁-C₆)-alkyl”.

Analogously, “(C₁-C₆)-alkylsulfinyl” is, for example, the methyl-, ethyl-, propyl-, isopropyl-, butyl-, isobutyl-, sec-butyl- or tert-butylsulfinyl group and “(C₁-C₆)-alkylsulfonyl” is, for example, the methyl-, ethyl-, propyl-, isopropyl-, butyl-, isobutyl-, sec-butyl- or tert-butylsulfonyl group.

“(C₁-C₆)-alkylamino” is a nitrogen atom which is substituted by one or two identical or different alkyl radicals of the above definition.

The term “(C₁-C₆)-mono- or -dialkylcarbamoyl” denotes a carbamoyl group having one or two hydrocarbon radicals having the meaning given under the term “(C₁-C₆)-alkyl” which, in the case of two hydrocarbon radicals, may be identical or different.

Analogously, “(C₁-C₆)-dihaloalkylcarbamoyl” is a carbamoyl group which carries two (C₁-C₆)-haloalkyl radicals of the above definition or one (C₁-C₆)-haloalkyl radical and one (C₁-C₆)-alkyl radical of the above definition.

“(C₁-C₆)-alkanoyl” is, for example, the formyl, acetyl, propionyl, butyryl or 2-methylbutyryl group.

The term “(C₆-C₁₄)-aryl” is to be understood as meaning a carbocyclic radical, i.e. an aromatic radical constructed of carbon atoms, having 6 to 14, in particular 6 to 12, carbon atoms, such as, for example, phenyl, naphthyl or biphenyl, preferably phenyl.

Accordingly, “aroyl” is an aryl radical as defined above which is attached via a carbonyl group, such as, for example, the benzoyl group.

The term “(C₃-C₁₀)-heterocyclyl” denotes a cyclic radical which has 3 to 10 carbon atoms, which may be fully saturated, partially unsaturated or fully unsaturated or aromatic and which is interrupted by one or more identical or different atoms from the group consisting of nitrogen, sulfur and oxygen, where, however, two oxygen atoms may not be directly adjacent and at least one carbon atom must still be present in the ring, such as, for example, a thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine, 4H-quinolizine, piperidine, pyrrolidine, oxazoline, tetrahydrofuran, tetrahydropyrane, isoxazolidine, isoxazoline, thiazoline, morpholine, piperazine, oxirane, oxetane or thiazolidine radicals. Accordingly, from among the meanings mentioned above under “heterocyclyl”, the term “heteroaromatic” encompasses in each case the completely unsaturated aromatic heterocyclic compounds.

Heterocyclyl is preferably a saturated, partially saturated or aromatic ring system having 3 to 6 ring members and 1 to 4 heteroatoms from the group consisting of O, S and N, where at least one carbon atom must be present in the ring.

Heterocyclyl is very particularly preferably a pyridine, pyrimidine, (1,2,4)-oxadiazole, (1,3,4)-oxadiazole, pyrrole, furan, thiophene, oxazole, thiazole, imidazole, pyrazole, isoxazole, 1,2,4-triazole, tetrazole, pyrazine, pyridazine, oxazoline, thiazoline, tetrahydrofuran, tetrahydropyrane, morpholine, piperidine, piperazine, pyrroline, pyrrolidine, oxazolidine, thiazolidine, oxirane and oxetane radical.

“Aryl-(C₁-C₄)-alkoxy” denotes an aryl radical which is attached via a (C₁-C₄)-alkoxy group, for example the benzyloxy, phenylethoxy, phenylbutoxy or naphthylmethoxy radical.

“Arylthio” is an aryl radical which is attached via a sulfur atom, for example the phenylthio or the 1- or 2-naphthylthio radical. Analogously, “aryloxy” is, for example, the phenoxy or 1- or 2-naphthyloxy radical.

“Aryl-(C₁-C₄)-alkylthio” is an aryl radical which is attached via an alkylthio radical, for example the benzylthio, naphthylmethylthio or the phenylethylthio radical.

The term “(C₁-C₆)-trialkylsilyl” denotes a silicon atom which carries three identical or different alkyl radicals having one to six carbon atoms, according to the above definition. Analogously, “aryl-(C₁-C₆)-dialkylsilyl” denotes a silicon atom which carries one aryl radical and two identical or different alkyl radicals having one to six carbon atoms, according to the above definition, “diaryl-(C₁-C₆)-alkylsilyl” denotes a silicon atom which carries one alkyl radical having one to six carbon atoms and two identical or different aryl radicals, according to the above definition, and “triarylsilyl” denotes a silicon atom which carries three identical or different aryl radicals, according to the above definition.

Preferred substitutents which may be present on various aliphatic, aromatic and heterocyclic ring systems include halogen, nitro, cyano, di-(C₁-C₄)-alkylamino, (C₁-C₄)-alkyl, (C₁-C₄)-trialkylsilyl, (C₁-C₄)-alkoxy, (C₁-C₄)-alkoxy-(C₁-C₄)-alkyl, (C₁-C₂)-alkoxy-[CH₂CH₂]_{1,2}-ethoxy, (C₁-C₄)-alkylthio, (C₁-C₄)-alkylsulfinyl, (C₁-C₄)-alkylsulfonyl, phenyl, benzyl, phenoxy, phenylthio, halophenoxy, (C₁-C₄)-alkylthiophenoxy, (C₁-C₄)-alkoxyphenoxy, (C₁-C₄)-alkylthiophenoxy, phenylthio, heterocyclyl, heterocyclylthio, heterocyclyloxy, haloheterocyclyloxy, alkylheterocyclyloxy or alkoxyheterocyclyloxy, where in the alkyl radicals and the radicals derived therefrom one or more hydrogen atoms, in the case of fluorine also up to the maximum number, may be replaced by halogen, preferably chlorine or fluorine.

Particularly preferred substituents are, in particular in the case of cyclic systems, halogen, cyano, nitro, amino, hydroxy, thio, (C₁-C₄)-alkyl, (C₁-C₄)-haloalkyl, (C₃-C₈)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylamino, (C₁-C₄)-haloalkylamino, formyl and (C₁-C₄)-alkanoyl.

Depending on the nature of the substituents defined above, the compounds of the formula (I) have acidic or basic properties and may be able to form salts. If the compounds of the formula (I) carry, for example, groups like hydroxyl, carboxyl or other groups which induce acidic properties, these compounds may be reacted with bases to form salts. Suitable bases are, for example, hydroxides, carbonates, bicarbonates of the alkali metals and alkaline earth metals, in particular those of sodium, potassium, magnesium and calcium, furthermore ammonia, primary, secondary and tertiary amines with (C₁-C₄)-alkyl radicals and also mono-, di- and trialkanolamines of (C₁-C₄)-alkanols. If the compounds of the formula (I) carry, for example, groups such as amino, alkylamino or other groups which induce basic properties, these compounds can be reacted with acids to form salts. Suitable acids, for example mineral acids, such as hydrochloric acid, sulfuric acid and phosphoric acid, organic acids, such as acetic acid or oxalic acid, and acidic salts, such as NaHSO₄ and KHSO₄. The salts obtainable in this manner likewise have insecticidal, acaricidal and miticidal properties.

The compounds of the formula (I) can have an asymmetric sulfur atom and/or one or more asymmetric carbon atoms or stereoisomers on double bonds. Therefore, it is possible for enantiomers or diastereomers to be present. The invention embraces both the pure isomers and their mixtures. The mixtures of diastereomers can be separated into the isomers by customary methods, for example by selective crystallization from suitable solvents or by chromatography. Racemates can be separated into the enantiomers by customary methods.

The preparation of the compounds according to the invention can be carried out under reaction conditions which are known and suitable for the reactions mentioned. It is also possible to use variants which are known per se but not mentioned here in detail. If desired, the starting materials can also be formed in situ, i.e. they are not isolated from the reaction mixture but immediately reacted further to give the compounds of the formula (I).

Hydroxamic acid derivatives of the formula (I) in which R¹, R², R³, R⁴, R⁵, X, Y and n are as defined above can be obtained by reacting activated acid derivatives of the formula (II) with hydroxylamine derivatives of the formula (III) according to the scheme below. Z in the formula (II) may be a halogen, such as chlorine or bromine.

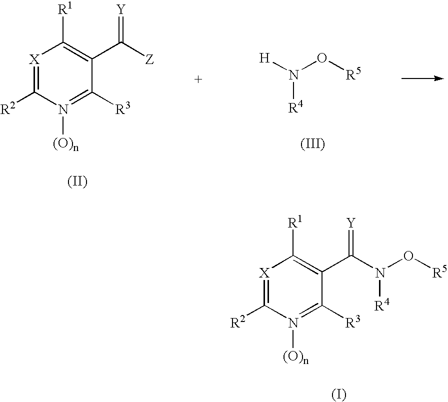

The compounds according to the invention can also be obtained by derivatizing the unsubstituted hydroxamic acid ((I); R⁴ and R⁵ are hydrogen) or by substitution, if R⁴ or R⁵ is hydrogen.

General procedures for preparing substituted hydroxamic acids can be found in D. Döpp and H. Döpp in Houben-Weyl, Methoden der organischen Chemie [Methods of Organic Chemistry], 4th edition, volume E5 II, p. 1141, Thieme Verlag, Stuttgart, 1984.

The acid ((II); Z=hydroxyl) may be activated by conversion into the acid halide ((II), Z=halide). Possible halogenating agents are, for example, oxalyl chloride, POCl₃, PCl₃, PCl₅, SOCl₂ or SO₂Cl₂.

The acid halides obtained in this manner can then be reacted further with hydroxylamine ((III), R⁴, R⁵=hydrogen) or else with hydroxylamine derivatives (III) (literature: Jenks, J. Amer. Chem. Soc. (1958), 80, 4585; Beckmann, Chem. Ber. (1993), 26, 2625). In a typical method, the acid halide ((II); Z=halide) is reacted with hydroxylamine derivatives (III) in the presence of a base. Suitable bases are, for example, alkali metal or alkaline earth metal hydroxides, carbonates or bicarbonates or organic bases, such as, for example, trialkylamines or pyridines. It is also possible to use bases attached to a solid phase, such as, for example, S-Trisamine from Agilent or Polystyrene AM NH2 from Rapp. The reaction is advantageously carried out in an inert solvent, such as, for example, dichloromethane, chloroform, carbon tetrachloride, benzene, toluene, diethyl ether or tetrahydrofuran, or else in mixtures of these solvents, in a temperature range between 0° C. and 100° C., preferably from 20° C. to 50° C.

Alternatively, the acid can also be reacted directly with hydroxylamine derivatives by employing coupling agents, such as carbonyldiimidazol (CDI), dicyclohexyl-carbodiimide (DCC) or N-ethyl-N′-(3-dimethylaminopropyl)carbodiimide hydrochloride (EDAC) (literature: D. Geffken, G. Zimmer, Chem. Ber. (1973) 106, 2246; M. Miller et al., J. Amer. Chem. Soc. (1980), 120, 7026). Also possible is a direct reaction of the esters ((II), X═OR) with the hydroxylamine derivatives (Ill).

Substituted hydroxamic acids in which R⁵ is hydrogen can also be obtained, for example, by hydrogenating compounds (I) in which R⁵ is a benzylic substituent, for example with hydrogen.

The resulting substituted hydroxamic acids (I) in which R⁴ or R⁵ or R⁴ and R⁵ are hydrogen can be converted into the target compounds (I) by alkylation or acylation according to methods known per se (as described, for example, in R. C. Larock, Comprehensive Organic Transformations, VCH Weinheim). In a typical method, the hydroxamic acid (I, R⁴ or R⁵ or R⁴ and R⁵ are hydrogen) is reacted with acid chlorides in the presence of a base. Suitable bases are, for example, alkali metal or alkaline earth metal hydroxides, carbonates or bicarbonates or organic bases, such as, for example, trialkylamines or pyridines. It is also possible to use bases attached to solid supports, such as the examples mentioned above. The reaction is advantageously carried out in an inert solvent, at temperatures between 0° C. and 100° C., preferably between 20° C. and 50° C. Examples of inert solvents are listed further above.

The invention also relates to a process for preparing the above-defined compounds of the formula (I), 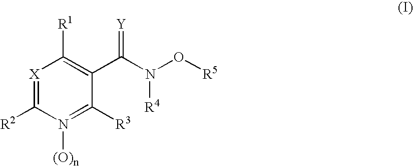
in which R¹, R², R³, R⁴, R⁵, X, Y and n are as defined above further and at least one of the radicals R⁴ or R⁵ is —C(W)R⁸, —C(W)OR⁸, —C(W)SR⁸, —C(W)NR⁸₂, —C(W)NR⁸—NR⁸₂, —C(W)NR⁸—NR⁸[C(W)R⁸], —SO₂NR⁸₂, —SO₂OR⁸, —S(O)R⁸, —S(O)₂R⁸, —PW₂R⁸ or —PWR⁸, by reacting a compound of the formula (I) in which R⁴ and R⁵ are hydrogen with a compound of the formula (IV)
R¹²-Hal (IV)
in which Hal is a halogen atom and R¹² is a radical selected from the group consisting of —C(W)R⁸, —C(W)OR⁸, —C(W)SR⁸, —C(W)NR⁸₂, —C(W)NR⁸—NR⁸₂, —C(W)NR⁸—NR⁸[C(W)R⁸], —SO₂NR⁸², —SO₂OR⁸, —S(O)R⁸, —S(O)₂R⁸, —PWR⁸₂ and —PW(OR⁸)₂, where W and R⁸ are as defined further above.

To prepare compounds of the formula (I) in which n is 1, the pyridine nitrogen may be oxidized (see, for example, Houben-Weyl, 4th edition (1992), Vol. E 7b, G. Thieme, Stuttgart). Suitable oxidizing agents are, for example, organic peracids, such as 3-chloroperbenzoic acid, and hydrogen peroxide.

The acid of the formula (II) in which X is —CH═ and R¹ is CF₃ has been characterized as a substance and is commercially available, for example from Aldrich (Steinheim, Germany).

Other pyridines and also the pyrimidines, can be prepared by methods known from the literature (see, for example, D. Spitzner “Methoden der Organischen Chemie”, Houben-Weyl, 4th edition (1992) Vol. E 7b, G. Thieme, Stuttgart; M. G. Hoffmann, A. Nowak, M. Müller, Houben-Weyl, 4th edition (1998), Vol. E 9b/Part 1).

The processes according to the invention are preferably suitable for carrying out the reactions in parallel.

Collections of compounds of the formula (I) which can be synthesized by the abovementioned scheme may also be prepared in a parallel manner, and this may be effected manually or in a semiautomated or fully automated manner. In this case, it is possible, for example, to automate the procedure of the reaction, work-up or purification of the products or of the intermediates. In total, this is to be understood as meaning a procedure as is described, for example, by S. H. DeWitt in “Annual Reports in Combinatorial Chemistry and Molecular Diversity: Automated Synthesis”, Volume 1, Verlag Escom 1997, pages 69 to 77.

A series of commercially available apparatuses as are offered by, for example, Stem Corporation, Woodrolfe Road, Tollesbury, Essex, CM9 8SE, England or H+ P Labortechnik GmbH, Bruckmannring 28, 85764 Oberschleiβheim, Germany or by Radleys, Shirehill, Saffron Walden, Essex, England may be used for the parallel procedure of the reaction and work-up. For the parallel purification of compounds of the formula (I), or of intermediates obtained during the preparation, use may be made, inter alia, of chromatography apparatuses, for example those from ISCO, Inc., 4700 Superior Street, Lincoln, Nebr. 68504, USA.

The apparatuses mentioned lead to a modular procedure in which the individual process steps are automated, but manual operations must be performed between the process steps. This can be prevented by employing semi-integrated or fully integrated automation systems where the automation modules in question are operated by, for example, robots. Such automation systems can be obtained, for example, from Zymark Corporation, Zymark Center, Hopkinton, Mass. 01748, USA.

In addition to the methods described here, compounds of the formula (I) may be prepared in part or fully by solid-phase-supported methods. For this purpose, individual intermediate steps or all intermediate steps of the synthesis or of a synthesis adapted to suit the procedure in question are bound to a synthetic resin. Solid-phase-supported synthesis methods are described extensively in the specialist literature, for example Barry A. Bunin in “The Combinatorial Index”, Academic Press, 1998.

The use of solid-phase-supported synthesis methods permits a series of protocols which are known from the literature and which, in turn, can be performed manually or in an automated manner. For example, the “tea-bag method” (Houghten, U.S. Pat. No. 4,631,211; Houghten et al., Proc. Natl. Acad. Sci, 1985, 82, 5131-5135), in which products from IRORI, 11149 North Torrey Pines Road, La Jolla, Calif. 92037, USA are employed, may be semiautomated. The automation of solid-phase-supported parallel syntheses is performed successfully, for example, by apparatuses from Argonaut Technologies, Inc., 887 Industrial Road, San Carlos, Calif. 94070, USA or MultiSynTech GmbH, Wullener Feld 4, 58454 Witten, Germany.

The preparation by the processes described herein yields compounds of the formula (I) in the form of substance collections which are termed libraries.

The present invention also relates to libraries which comprise at least two compounds of the formula (I).

The compounds of the formula (I) are suitable for controlling animal pests, in particular insects, arachnids, helminths and molluscs, very particularly preferably for controlling insects and arachnids which are encountered in agriculture, in livestock breeding, in forests, in the protection of stored goods and materials, and in the hygiene sector, and have good plant tolerance and favorable toxicity to warm-blooded species. They are active against normally sensitive and resistant species and against all or individual developmental stages. The abovementioned pests include:

From the order of the Acarina, for example, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Eotetranychus spp., Oligonychus spp., Eutetranychus spp.

From the order of the Isopoda, for example, Oniscus aselus, Armadium vulgare, Porcellio scaber.

From the order of the Diplopoda, for example, Blaniulus guttulatus.

From the order of the Chilopoda, for example, Geophilus carpophagus, Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Blafta orientalis, Periplaneta americana, Leucophaea maderae, Blaftella germanica, Acheta domesticus, Gryllotalpa spp., Locusta migratoria migratorioides, Melanoplus differentialis, Schistocerca gregaria.

From the order of the Isoptera, for example, Reticulitermes spp.

From the order of the Anoplura, for example, Phylloera vastatrix, Pemphigus spp., Pediculus humanus corporis, Haematopinus spp., Linognathus spp.

From the order of the Mallophaga, for example, Trichodectes pp., Damalinea spp.

From the order of the Thysanoptera, for example, Hercinothrips femoralis, Thrips tabaci.

From the order of the Heteroptera, for example, Eurygaster spp., Dysdercus intermedius, Piesma quadrata, Cimex lectularius, Rhodnius prolixus, Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Doralis fabae, Doralis pomi, Eriosoma lanigerum, Hyalopterus arundinis, Macrosiphum avenae, Myzus spp., Phorodon humuli, Rhopalosiphum padi, Empoasca spp., Euscelus bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp.

From the order of the Lepidoptera, for example, Pectinophora gossypiella, Bupalus piniarius, Cheimatobia brumata, Lithocolletis blancardella, Hyponomeuta padella, Plutella maculipennis, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Laphygma exigua, Mamestra brassicae, Panolis flammea, Prodenia litura, Spodoptera spp., Trichoplusia ni, Carpocapsa pomonella, Pieris spp., Chilo spp., Pyrausta nubilalis, Ephestia kuehniella, Galleria mellonella, Cacoecia podana, Capua reticulana, Choristoneura fumiferana, Clysia ambiguella, Homona magnanima, Tortrix viridana.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylloides chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrynchus assimilis, Hypera postica, Dermestes spp., Trogoderma, Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica.

From the order of the Hymenoptera, for example, Diprion spp., Hoplocampa spp., Lasius spp., Monomorium pharaonis, Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hypobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa.

From the order of the Siphonaptera, for example, Xenopsylla cheopsis, Ceratophyllus spp.

From the order of the Arachnida, for example, Scorpio maurus, Latrodectus mactans.

From the class of the helminths, for example, Haemonchus, Trichostrongulus, Ostertagia, Cooperia, Chabertia, Strongyloides, Oesophagostomum, Hyostrongulus, Ancylostoma, Ascaris and Heterakis and also Fasciola.

From the class of the Gastropoda, for example, Deroceras spp., Arion spp., Lymnaea spp., Galba spp., Succinea spp., Biomphalaria spp., Bulinus spp., Oncomelania spp. From the class of the Bivalva, for example, Dreissena spp.

It is furthermore possible to control protozoa, such as Eimeria.

The plant-parasitic nematodes which can be controlled in accordance with the invention include, for example, the root-parasitic soil-dwelling nematodes such as, for example, those of the genera Meloidogyne (root knot nematodes, such as Meloidogyne incognita, Meloidogyne hapla and Meloidogyne javanica), Heterodera and Globodera (cyst-forming nematodes, such as Globodera rostochiensis, Globodera pallida, Heterodera trifolii) and of the genera Radopholus, such as Radopholus similis, Pratylenchus such as Pratylenchus neglectus, Pratylenchus penetrans and Pratylenchus curvitatus; Tylenchulus such as Tylenchulus semipenetrans, Tylenchorhynchus, such as Tylenchorhynchus dubius and Tylenchorhynchus claytoni, Rotylenchus such as Rotylenchus robustus, Heliocotylenchus such as Haliocotylenchus multicinctus, Belonoaimus such as Belonoaimus longicaudatus, Longidorus such as Longidorus elongatus, Trichodorus such as Trichodorus primitivus and Xiphinema such as Xiphinema index.

Nematode genera which can furthermore be controlled using the compounds according to the invention are Ditylenchus (stem parasites, such as Ditylenchus dipsaci and Ditylenchus destructor), Aphelenchoides (foliar nematodes, such as Aphelenchoides ritzemabosi) and Anguina (flower nematodes, such as Anguina tritici).

The compounds according to the invention are preferably suitable for controlling sucking insects, such as aphids (for example Aphis fabae, Aphis pomi, Aphis spiraecola, Aphis gossypii, Aphis nasturtii, Dysaphis plantaginea, Eriosoma spp., Rhopalosiphum padi, Acyrthosiphon pisum, Pemphigus bursarius, Myzus persicae, Myzus nicotianae, Myzus euphorbiae, Phylloxera spp., Toxoptera spp, Brevicoryne brassicae, Macrosiphum avenae, Macrosiphum euphorbiae, Nasonovia ribisnigri, Sitobion avenae, Brachycaudus helychrysii or Phorodon humuli), cicadas (Idioscopus clypealis, Scaphoides titanus, Empoasca onuki, Empoasca vitis, Empoasca devastans, Empoasca libyca, Empoasca biguftula, Empoasca facialis, or Erythroneura spp), Thrips (Hercinothrips femoralis, Scirtothrips aurantii, Scirtothrips dorsalis, Frankliniella schultzei, Frankliniella fusca, Frankliniella occidentalis, Frankliniella tritici, Kakothrips spp., Thrips oryzae, Thrips palmi, Thrips tabaci) or white flies (Aleurodes brassicae, Bemisia tabaci, Trialeurodes vaporariorum, Aleurodes proletella).

The invention also relates to compositions, for example crop protection compositions, preferably insecticidal, acaricidal, ixodicidal, nematicidal, molluscicidal or fungicidal, especially preferably insecticidal and acaricidal, compositions which comprise one or more compounds of the formula (I) in addition to suitable formulation auxiliaries.

In general, the compositions according to the invention comprise 1 to 95% by weight of the active compounds of the formula (I).

To prepare the compositions according to the invention, the active compound and the other additives are combined and brought into a suitable use form.

The compositions according to the invention comprise 1 to 95% by weight of the active compounds of the formula (I). They can be formulated in various ways, depending on the biological and/or chemical-physical parameters which prevail. The following are examples of possible formulations:

Wettable powders (WP), emulsifiable concentrates (EC), aqueous solutions (SL), emulsions, sprayable solutions, oil- or water-based dispersions (SC), suspo-emulsions (SE), dusts (DP), seed-dressing products, granules in the form of microgranules, spray granules, coated granules and adsorption granules, water-dispersible granules (WG), ULV formulations, microcapsules, waxes or baits.

These individual types of formulations are known in principle and are described, for example, in: Winnacker-Küchler, “Chemische Technologie” [Chemical Technology], Volume 7, C. Hanser Verlag Munich, 4th Edition 1986; van Falkenberg, “Pesticides Formulations”, Marcel Dekker N.Y., 2nd Ed. 1972-73; K. Martens, “Spray Drying Handbook”, 3rd Ed. 1979, G. Goodwin Ltd. London.

The necessary formulation auxiliaries, i.e. carrier materials and/or surface-active compounds such as inert materials, surfactants, solvents and other additives, are also known and described, for example, in: Watkins, “Handbook of Insecticide Dust Diluents and Carriers”, 2nd Ed., Darland Books, Caldwell N.J.; H. v. Olphen, “Introduction to Clay Colloid Chemistry”, 2nd Ed., J. Wiley & Sons, N.Y.; Marsden, “Solvents Guide”, 2nd Ed., Interscience, N.Y. 1950; McCutcheon's, “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Surface-active ethylene oxide adducts], Wiss. Verlagsgesell., Stuttgart 1967; Winnacker-Küchler, “Chemische Technologie”, Volume 7, C. Hanser Verlag Munich, 4th Edition 1986.

Based on these formulations, it is also possible to prepare combinations with other pesticidally active materials, fertilizers and/or growth regulators, for example in the form of a ready-mix formulation or a tank mix. Wettable powders are preparations which are uniformly dispersible in water which, besides the active compound, also comprise wetters, for example polyoxyethylated alkylphenols, polyoxyethylated fatty alcohols, alkylsulfonates or alkylphenolsulfonates and dispersants, for example sodium lignosulfonate or sodium 2,2′-dinaphthylmethane-6,6′-disulfonate, in addition to a diluent or inert material.

Emulsifiable concentrates are prepared by dissolving the active compound in an organic solvent, for example butanol, cyclohexanone, dimethylformamide, xylene or else higher-boiling aromatics or hydrocarbons, with addition of one or more emulsifiers. As emulsifiers, the following can be used, for example: calcium alkylarylsulfonates such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensates, alkyl polyethers, sorbitan fatty acid esters, polyoxyethylene sorbitan fatty acid esters or polyoxyethylene sorbitol esters.

Dusts are obtained by grinding the active compound with finely divided solid materials, for example talc or natural clays, such as kaolin, bentonite, pyrophyllite or diatomaceous earth. Granules can be prepared either by atomizing the active compound onto adsorptive, granulated inert material or by applying active compound concentrates onto the surface of carrier materials such as sand or kaolinites, or of granulated inert material, by means of adhesives, for example polyvinyl alcohol or sodium polyacrylate, or else mineral oils. Suitable active compounds can also be granulated in the manner which is customary for the preparation of fertilizer granules, if desired as a mixture with fertilizers.

The active compound concentration in wettable powders is usually approximately 10 to 90% by weight, the remainder to 100% by weight is composed of customary formulation constituents. In the case of emulsifiable concentrates, the active compound concentration may be approximately 5 to 80% by weight. Formulations in the form of dusts usually comprise 5 to 20% by weight of active compound, sprayable solutions approximately 2 to 20% by weight. In the case of granules, the active compound content depends partly on whether the active compound is in liquid or solid form and on which granulation auxiliaries, fillers and the like are being used.

Besides this, the abovementioned active compound formulations comprise, if appropriate, the tackifiers, wetters, dispersants, emulsifiers, penetrants, solvents, fillers or carriers which are conventional in each case.

For use, the concentrates, which are present in commercially available form, are, if desired, diluted in the customary manner, for example in the case of wettable powders, emulsifiable concentrates, dispersions and in some cases also microgranules, using water. Preparations in the form of dusts and granules and sprayable solutions are usually not diluted any further with other inert substances prior to use.

The application rate required varies with the external conditions such as, inter alia, temperature and humidity. It may vary within wide limits, for example between 0.0005 and 10.0 kg/ha or more of active compound, but it is preferably between 0.001 and 5 kg/ha of active compound.

The active compounds according to the invention, in their commercially available formulations and in the use forms prepared from these formulations, may be present in mixtures with other active compounds such as insecticides, attractants, sterilants, acaricides, nematicides, fungicides, growth-regulating substances or herbicides.

The invention therefore also provides a composition having insecticidal, acaricidal, ixodicidal, nematicidal and/or molluscidal action, which composition comprises, in addition to a compound of the formula (I), at least one further active compound from the group of the insecticides, acaricides, nematicides, fungicides, herbicides and growth-regulating substances.

The pesticides include, for example, phosphoric esters, carbamates, carboxylic esters, formamidines, tin compounds and materials produced by microorganisms.

Preferred components in mixtures are:
1. from the group of the phosphorus compounds acephate, azamethiphos, azinphos-ethyl, azinphos-methyl, bromophos, bromophos-ethyl, cadusafos (F-67825), chlorethoxyphos, chlorfenvinphos, chlormephos, chlorpyrifos, chlorpyrifos-methyl, demeton, demeton-S-methyl, demeton-S-methyl sulfone, dialifos, diazinon, dichlorvos, dicrotophos, dimethoate, disulfoton, EPN, ethion, ethoprophos, etrimfos, famphur, fenamiphos, fenitriothion, fensulfothion, fenthion, flupyrazofos, fonofos, formothion, fosthiazate, heptenophos, isazophos, isothioate, isoxathion, malathion, methacrifos, methamidophos, methidathion, salithion, mevinphos, monocrotophos, naled, omethoate, oxydemeton-methyl, parathion, parathion-methyl, phenthoate, phorate, phosalone, phosfolan, phospho-carb (BAS-301), phosmet, phosphamidon, phoxim, pirimiphos, pirimiphos-ethyl, pirimiphos-methyl, profenofos, propaphos, proetamphos, prothiofos, pyraclofos, pyridapenthion, quinalphos, suiprofos, temephos, terbufos, tebupirimfos, tetra-chlorvinphos, thiometon, triazophos, trichlorphon, vamidothion;
2. from the group of the carbamates alanycarb (OK-135), aldicarb, 2-sec-butylphenyl methylcarbamate (BPMC), carbaryl, carbofuran, carbosulfan, cloethocarb, benfuracarb, ethiofencarb, furathiocarb, HCN-801, isoprocarb, methomyl, 5-methyl-m-cumenylbutyryl (methyl)carbamate, oxamyl, pirimicarb, propoxur, thiodicarb, thiofanox, 1-methylthio(ethylideneamino)-N-methyl-N-(morpholinothio)carbamate (UC 51717), triazamate;
3. from the group of the carboxylic esters acrinathrin, allethrin, alphametrin, 5-benzyl-3-furylmethyl (E)-(1R)-cis-2,2-dimethyl-3-(2-oxothiolan-3-ylidenemethyl)cyclopropanecarboxylate, beta-cyfluthrin, alpha-cypermethrin beta-cypermethrin, bioalleth rin, bioallethrin ((S)-cyclopentylisomer), bioresmethrin, bifenthrin, (RS)-1-cyano-1-(6-phenoxy-2-pyridyl)methyl (1RS)-trans-3-(4-tert-butylphenyl)-2,2-dimethylcyclopropanecarboxylate (NCI 85193), cycloprothrin, cyfluthrin, cyhalothrin, cythithrin, cypermethrin, cyphenothrin, deltamethrin, empenthrin, esfenvalerate, fenfluthrin, fenpropathrin, fenvalerate, flubrocythrinate flucythrinate, flumethrin, fluvalinate (D isomer), imiprothrin (S-41311), lambda-cyhalothrin, permethrin, phenothrin ((R) isomer), prallethrin, pyrethrins (natural products), resmethrin, tefluthrin, tetramethrin, theta-cypermethrin, tralomethrin, transfluthrin, zeta-cypermethrin (F-56701);
4. from the group of the amidines amitraz, chlordimeform;
5. from the group of the tin compounds cyhexatin, fenbutatin oxide;
6. others Abamectin, ABG-9008, acequinocyl, acetamiprid, Anagrapha falcitera, AKD-1022, AKD-3059, AKD-3088, AL-9811, ANS-118, azadirachtin, Bacillus thuringiensis, Beauveria bassianea, bensultap, bifenazate (D-2341), binapacryl, BJL-932, bromopropylate, BAJ-2740 (spirodiclofen), BSN 2060 (spiromesifen), BTG-504, BTG-505, buprofezin, camphechlor, cartap, chlorobenzilate, chlorfenapyr, chlorfluazuron, 2-(4-chlorophenyl)-4,5-diphenylthiophene (UBI-T 930), chlorfentezine, chloproxyfen, chromafenozide ANS-118), A-184699, clothianidine, 2-naphthylmethyl cyclopropancarboxylate (Ro12-0470), cyromazine CM-002×, DBI-3204, diacloden (thiamethoxam), diafenthiuron, DBI-3204, ethyl N-(3,5-dichloro-4-(1,1,2,3,3,3-hexafluoro-1-propyloxy)phenyl)carbamoyl)-2-chlorobenzocarboximidate, D DT, d icofol, diflubenzuron, N-(2,3-dihydro-3-methyl-1,3-thiazol-2-ylidene)-2,4-xylidine, dihydroxymethyldihydroxypyrrolidine, dinobuton, dinocap, diofenolan, DPX-062 emarnectin-benzoate (MK-244), endosulfan, ethiprole (sulfethiprole), ethofenprox, etoxazole (YI-5301), fenazaquin, fenoxycarb, fipronil, flonicamid (IKI 220), fluazuron, flumite (flufenzine, SZI-121), 2-fluoro-5-(4-(4-ethoxyphenyl)₄-methyl-1-pentyl)diphenylether (MTI 800), granulosis and nuclear polyhedrosis viruses, fenpyroximate, fenthiocarb, fluacrypyrim, flubenzimine, flubrocythrinate, flucycloxuron, flufenoxuron, flufenprox (IC1-A5683), flufenzine, fluproxyfen, FMC-F6028, gamma-HCH, halofenozide (RH-0345), halofenprox (MTI-732), hexaflumuron (DE_{—}473), hexythiazox, HOI-9004, hydramethylnon (AC 217300), lufenuron, L-14165 imidacloprid, indoxacarb (DPX-MP062), kanemite (AKD-2023), M-020, MTI-466, ivermectin, IKA-2000 MKI-245, methoxyfenozide (Intrepid, RH-2485), milbemectin, NC-196, neemgard, nitenpyram (TI-304), 2-nitromethyl-4,5-dihydro-6H-thiazine (DS 52618), 2-nitromethyl-3,4-dihydrothiazole (SD 35651), 2-nitromethylene-1,2-thiazinan-3-ylcarbamaldehyde (WL 108477), novaluron, NC-196, NNI-0001, nidintefuran, propargite, pyriproxyfen (S-71639), pirydaryl, protrifenbute, pyriproxyfen, NC-196, NC-1111, NNI-9768, novaluron (MCW-275), OK-9701, OK-9601, OK-9602, OK-9802, propargite, pymethrozine, pyridaben, pyrimidifen (SU-8801), R-195, RH-0345, RH-2485, RYI-210, S-1283, S-1833, SB7242, SI-8601, silafluofen, silomadine (CG-177), spinosad, spirodiclofen, spiromesifen, SU-9118, tebufenozide, tebufenpyrad (MK-239), teflubenzuron, tetradifon, tetrasul, thiacloprid, thiocyclam, thiamethoxam, Tl435, tolfenpyrad (OMI-88), triazamate (RH-7988), triflumuron, triethoxyspinosyn A, verbutin, vertalec (Mykotal), YI-5301 and Yi-6101.

The abovementioned components for combinations are known active compounds, many of which are described in Ch. R Worthing, S. B. Walker, The Pesticide Manual, 12th Edition, British Crop Protection Council, Farnham 2000.

The following products, for example, may be mentioned as fungicides which can be combined with the compounds of the formula (I) according to the invention:

Aldimorph, andoprim, anilazine, BAS 480F, BAS 450F, benalaxyl, benodanil, benomyl, binapacryl, bitertanol, bromuconazole, buthiobate, captafol, captan, carbendazim, carboxin, CGA 173506, cyprofuram, dichlofluanid, dichlomezin, diclobutrazole, diethofencarb, difenconazole (CGA 169374), difluconazole, dimethirimol, dimethomorph, diniconazole, dinocap, dithianon, dodemorph, dodine, edifenfos, ethirimol, etridiazot, fenarimol, fenfuram, fenpiclonil, fenpropidin, fenpropimorph, fentin acetate, fentin hydroxide, ferimzone (TF164), fluazinam, fluobenzimine, flouxastrobin, fluquinconazole, fluorimide, flusilazole, flutolanil, fluthafol, folpet, fosetyl aluminum, fuberidazole, fulsulfamide (MT-F 651), furalaxyl, furconazole, furmecyclox, guazatine, hexaconazole, ICI A5504, Imazalil, Imibenconazole, iprobenfos, iprodione, isoprothiolane, KNF 317, copper compounds, such as Cu oxychloride, oxine-Cu, Cu oxide, mancozeb, maneb, mepanipyrim (KIF 3535), metconazole, mepronil, metalaxyl, methasulfocarb, methfuroxam, MON 24000, myclobutanil, nabam, nitrothalidopropyl, nuarimol, ofurace, oxadixyl, oxycarboxin, penconazole, pencycuron, PP 969, probenazole, propineb, prochloraz, procymidone, propamocarb, propiconazole, prothiocarb, pyracarbolid, pyraclostrobin, pyrazophos, pyrifenox, pyroquilon, rabenzazole, RH7592, sulfur, tebuconazole, TF 167, thiabendazole, thicyofen, thiofanatemethyl, thiram, tolclofos-methyl, tolylfluanid, triadimefon, triadimenol, tricyclazole, tridemorph, triflumizole, triforine, validamycin, vinchlozolin, XRD 563, zineb, sodium dodecylsulfonate, sodum dodecylsulfate, sodium C13/C15-alcohol ether sulfonate, sodium cetostearyl phosphate ester, dioctyl sodium sulfosuccinate, sodium isopropylnaphthalenesulfonate, sodium methylenebisnaphthalenesulfonate, cetyltrimethylammonium chloride, salts of long-chain primary, secondary or tertiary amines, alkylpropyleneamines, laurylpyrimidinium bromide, ethoxylated quarternized fatty amines, alkyldimethylbenzylammonium chloride and 1-hydroxyethyl-2-alkyl-imidazoline.

The active compound content of the use forms prepared from the commercially available formulations may range from 0.00000001 up to 95% by weight of active compound, preferably between 0.00001 and 1% by weight.

Application is effected in a customary manner adapted to suit the use forms. Accordingly, the invention also provides a method for controlling pests of plants, which method comprises treating the plants and/or the pests with an effective amount of a compound of the formula (I).

The invention also provides the use of a compound of the formula (I) for controlling pests of plants.

The active compounds according to the invention are also suitable for controlling endoparasites and ectoparasites in the human and veterinary medicine sector and/or in the field of animal keeping. The active compounds according to the invention are applied here in a known manner, such as by oral administration in the form of, for example, tablets, capsules, drinks or granules, by dermal application in the form of, for example, dipping, spraying, pouring on and spotting on, and dusting, and by parenteral administration in the form of, for example, an injection.

Accordingly, the compounds of the formula (I) according to the invention can also be employed particularly advantageously for the treatment of warm-blooded species, in particular in livestock keeping (for example cattle, sheep, pigs and poultry such as chickens, geese and the like). In a preferred embodiment of the invention, the compounds, if appropriate in suitable formulations, are administered orally to the animals, if appropriate together with the drinking water or feed. Since excretion in the feces is efficient, the development of insects in the animals' feces can be prevented very easily in this manner. The dosages and formulations which are suitable in each case depend, in particular, on the species and the developmental stage of the productive livestock and also on the risk of infestation and can be determined readily and established by customary methods. For example, the compounds can be employed in cattle at dosages of 0.01 to 1 mg/kg of bodyweight.

In addition to the abovementioned application methods, the active compounds of the formula (I) according to the invention have excellent systemic action. Accordingly, the active compounds can also be introduced into the plants via parts of the plant, both below ground and above ground (root, stem, leaf), if the active compounds are applied, in liquid or solid form into and/or in the direct vicinity of the plant (for example granules in soil application, application in flooded rice paddies, tree trunk injection, stalk bandages for perennial plants, stem painting, for example in the case of hops).

Furthermore, the active compounds according to the invention are particularly useful, optionally in co-formulation with fungicides, for the treatment of vegetative and generative plant propagation material, such as, for example, of seeds, for example of cereals, vegetables, cotton, rice, sugar beet and other crops and ornamental plants, of bulbs, seedlings and tubers of other crops and ornamental plants which are propagated vegetatively. The treatment can be carried out before sowing or before planting (for example by special seed coating techniques, by dressing in liquid or solid form or as a seed box treatment), during sowing or planting or after sowing or planting by special application techniques (for example furrow treatment). The amount of active compound used can vary within a relatively large range, depending on the application. In general, the application rates are between 1 g and 10 kg of active compound per hectare of soil surface. The treatment method for vegetable propagation material and the vegetable propagation material treated in this method are likewise provided by the invention.

The compounds of the formula (I) can also be employed for controlling weeds in crops of known genetically engineered plants or genetically engineered plants yet to be developed. As a rule, the transgenic plants are distinguished by especially advantageous properties, for example by resistances to particular crop protection agents, resistances to plant diseases or pathogens of plant diseases, such as particular insects or microorganisms such as fungi, bacteria or viruses. Other particular properties concern, for example, the harvested material with regard to quantity, quality, storage properties, composition and specific constituents. Thus, transgenic plants are known where the starch content is increased, or the starch quality is altered, or where the harvested material has a different fatty acid composition.

The use in economically important transgenic crops of useful plants and ornamentals is preferred, for example of cereals such as wheat, barley, rye, oats, millet, rice, cassaya and corn or else crops of sugar beet, cotton, soybean, oilseed rape, potatoes, tomatoes, peas and other types of vegetables.

When used in transgenic crops, in particular those which have resistances to insects, effects are frequently observed, in addition to the effects against harmful organisms to be observed in other crops, which are specific for application in the transgenic crop in question, for example an altered or specifically widened spectrum of pests which can be controlled, or altered application rates which may be employed for application.

The invention therefore also relates to the use of compounds of the formula (I) for controlling harmful organisms in transgenic crop plants, and to a method for controlling pests of transgenic plants, which method comprises treating the transgenic plant and/or the pests with an effective amount of a compound of the formula (I).

In addition to their lethal effect on pests, the compounds of the formula (I) also have a pronounced repellent effect.

A repellent for the purpose of the compound is a substance or substance mixture which has a warding-off or fending-off effect on other living beings, in particular harmful pests and nuisance pests. The term also encompasses effects such as the antifeeding effect, where the intake of feed is disturbed or prevented (antifeedant effect), suppression of oviposition, or an effect on the development of the population.

The invention therefore also provides the use of compounds of the formula (I) for achieving the abovementioned effects, in particular in the case of the pests stated in the biological examples.

The invention also provides a method for fending off, or warding off, harmful organisms, where one or more compounds of the formula (I) are applied to the site from which the harmful organisms are to be fended off or warded off.

In the case of a plant, application may mean, for example, a treatment of the plant, but also of the seed.

As regards the effect on populations, it is interesting to note that effects can also be observed in succession during the development of a population, where summation may take place. In such a case, the individual effect itself may only have an efficacy of markedly less than 100% but in total an efficacy of 100% is still achieved in the end.

Moreover, the compounds of the formula (I) are distinguished by the fact that the composition is usually applied earlier than in the case of a direct control, if the abovementioned effects are to be exploited. The effect frequently lasts over a long period, so that a duration of action of over 2 months is achieved.

The effects are observed in insects, arachnids and the other abovementioned pests.

The use of the compounds according to the invention comprises, in addition to direct application to the pests, any other application in which the compounds of the formula (I) act on the pests. Such indirect applications may, for example, be the use of compounds which decompose to compounds of the formula (I) or which are degraded to compounds of the formula (I), for example in the soil, in the plant or in the pest.

The content of the German patent application 102 39 905.0, the priority of which is claimed by the present application, and of the appended abstract is expressly incorporated into the present description by way of reference.

The examples below serve to illustrate the invention.

### CHEMICAL EXAMPLES

### Example 1

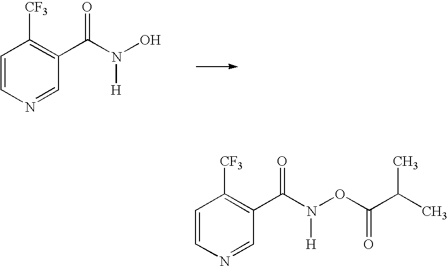

0.25 g (0.0012 mol) of 4-trifluoromethylnicotinhydroxamic acid was dissolved in 10 ml of dichloromethane. 0.17 ml (0.0012 mol) of triethylamine was added, and the reaction mixture was stirred at room temperature for 5 minutes. 0.13 ml (0.0012 mol) of isobutyryl chloride, dissolved in 3 ml of dichloromethane, was then added dropwise, and the solution was subsequently stirred at room temperature for 2 hours. The reaction was worked up by adding water and extracting with dichloromethane. After drying over MgSO₄, the extract was filtered and the solvent was removed. This gave 0.35 g of a clear oil. This was purified by chromatography using heptane and ethyl acetate. Yield: 0.2 g (58%) of a clear oil.

¹H-NMR 300 MHz (CDCl₃): 9.38 (br s 1H NH), 8.90 (m, 2H, 2-H pyridine), 7,66 (d, J=6 Hz, 1H, 5-H pyridine), 2.84 (m, 1H, CH), 1.32 (m, 6H, CH₃)

The compounds listed in the tables below were prepared in an analogous manner.

TABLE 1

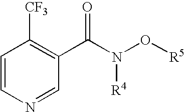

| Example | | | |
|---|---|---|---|
| No. | R⁴ | R⁵ | Note |
| 1 | H | —CO—CH(CH₃)₂ | Oil |
| 2 | —CO-(4-trifluoromethylpyrid-3-yl) | —CH₃ | Oil |
| 3 | —CO-(4-trifluoromethylpyrid-3-yl) | —C₂H₅ | Oil |
| 4 | H | —CO—CH₂—O—C₆H₅ | Oil¹ |
| 5 | —CO-(2-methoxyphenyl) | —CO-(2-methoxyphenyl) | Oil² |
| 6 | H | —CO-(2-chloro-4-fluorophenyl) | Oil³ |
| 7 | —CO-(2-chlorophenyl) | —CO-(2-chlorophenyl) | Oil |
| 8 | H | —CO-(2-chlorophenyl) | Oil⁴ |
| 9 | H | —CO-tert-butyl | Oil |
| 10 | —CO-cyclopropyl | —CO-cyclopropyl | Oil |
| 11 | —CO—CH═CH-CH₃ | —CO—CH═CH—CH₃ | Oil |
| 12 | H | —CO-(4-methylphenyl) | Oil⁵ |
| 13 | —CO-phenyl | —CO-phenyl | Oil⁶ |
| 14 | —CO—CH₃ | —CO—CH₃ | Oil⁷ |
| 15 | H | —CO—CH₃ | Oil |

¹¹H-NMR 300MHz (CDCl₃): 9.72 (brs 1H NH); 8.99 (m, 1H, 2-H pyridine); 8.61 (s, 1H, 6-H pyridine); 7.54 (d, J=6Hz, 1H, 5-H pyridine); 7.36 (tr, J=8Hz, 2H, 3′, 5′H phenyl); 7.08 (tr, J=8Hz, 1H, 4′H phenyl); 6.99 (tr, J=8Hz, 2H, 2′, 6′H phenyl); 4.68 (s, 2H, CH₂)
²¹H-NMR 300MHz (CDCl₃): 9.06 (s, 1H, 2-H pyridine); 8.84 (d, J=6Hz, 1H, 6-H pyridine); 7.65 (dd, J=6/2Hz, phenyl); 7.32-7.52 (m, 3H, phenyl); 6.80-6.96 (m, 3H, phenyl); 6.82 (d, J=8Hz, 1H, phenyl); 3.84 (s, 3H, CH₃); 3.79 (s, 2H, CH₂)
³¹H-NMR 300MHz (CDCl₃): 8.99 (s, 1H, 2-H pyridine); 8.83 (d, J=6Hz, 1H, 6-H pyridine); 8.12 (m, 1H, 6′-H phenyl); 7.61 (d, J=6Hz, 1H, 5H pyridine); 7.24 (m, 1H, 3′H phenyl); 7.08 (m, 1H, 5′H phenyl)
⁴¹H-NMR 300MHz (CDCl₃): 8.94 (s, 1H, 2-H pyridine); 8.88 (d, J=6Hz, 1H, 6-H pyridine); 8.08 (m, 1H, 6′-H phenyl); 7.66 (d, J=6Hz, 1H, 5H pyridine); 7.51 (m, 2H, 4′, 5′H phenyl); 7.39 (m, 4H, 2′, 6′H phenyl)
⁵¹H-NMR 300MHz (CDCl₃): 9.92 (br s, 1H, NH); 8.96 (s, 1H, 2-H pyridine); 8.90 (d, J=6Hz, 1H, 6-H pyridine); 8.02 (m, 2H, phenyl); 7.66 (d, J=6Hz, 1H, 5H pyridine); 7.30 (m, 2H, phenyl); 2.44 (s, 3H, CH₃)
⁶¹H-NMR 300MHz (CDCl₃): 9.38 (brs, 1H, NH); 8.90 (m, 2H, 6-H, 2-H pyridine); 7.66 (d, J=6Hz, 1H, 5-H pyridine); 2.84 (m, 1H, CH); 1.32 (s, 6H, CH₃)
⁷¹H-NMR 300MHz (CDCl₃): 8.98 (d, J=6Hz; 1H, 6-H pyridine); 8.63 (s, 1H, 2-H pyridine); 7.61 (d, J=6Hz, 1H, 5-H pyridine); 2.40 (s, 3H, CH₃); 2.20 (s, 3H, CH₃)

B. Formulation examples
a) A dust is obtained by mixing 10 parts by weight of active compound and 90 parts by weight of talc as inert material and comminuting the mixture in a hammer mill.
b) A wettable powder which is readily dispersible in water is obtained by mixing 25 parts by weight of active compound, 65 parts by weight of kaolin-containing quartz as inert material, 10 parts by weight of potassium ligno-sulfonate and 1 part by weight of sodium oleoylmethyltaurinate as wetter and dispersant and grinding the mixture in a pinned-disk mill.
   c) A dispersion concentrate which is readily dispersible in water is prepared by mixing 40 parts by weight of active compound with 7 parts by weight of a sulfosuccinic monoester, 2 parts by weight of a sodium lignosulfonate and 51 parts by weight of water and grinding the mixture in a ball mill to a fineness of below 5 microns.
   d) An emulsifiable concentrate can be prepared from 15 parts by weight of active compound, 75 parts by weight of cyclohexane as solvent and 10 parts by weight of oxyethylated nonylphenol (10 EO) as emulsifier.
   e) Granules can be prepared from 2 to 15 parts by weight of active compound and an inert granule carrier material such as attapulgite, pumice granules and/or quartz sand. It is expedient to use a suspension of the wettable powder of Example b) with a solids content of 30%, which is sprayed onto the surface of attapulgite granules, and these are dried and mixed intimately. The wettable powder amounts to approx. 5% by weight and the inert carrier material to approx. 95% by weight of the finished granules.
      C. Biological Examples

In the examples below, compounds were considered to be active when, at a concentration of 500 ppm (based on the content of active compound) or less, their effect on the harmful organisms was 50% or more.

Germinated field bean seeds (Vicia faba) with radicles were transferred into brown glass bottles filled with tap water and subsequently populated with approximately 100 black bean aphids (Aphis fabae). Plants and aphids were then dipped for 5 seconds into an aqueous solution of the formulated compound to be examined. After the solution had run off, plant and animals were stored in a climatized chamber (16 hours of light/day, 25° C., 40-60% relative atmospheric humidity). After 3 and 6 days of storage, the mortality effect of the compound on the aphids was determined. The compounds of the following examples were active: 1, 2, 5 to 11, 13 to 15.

1. A compound of the formula (I) or a salt thereof, 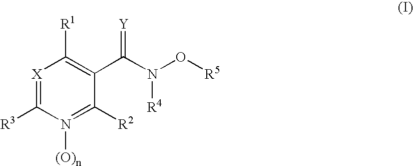
where the symbols and indices are as defined below:
X is CH or N;
Y is O or S;
n is 0 or 1;
R¹ is (C₁-C₆)-alkyl, (C₁-C₆)-haloalkyl, —S(halogen)₅ or halogen, where one or two CH₂ in the alkyl or haloalkyl groups is/are optionally replaced by —O— or —S— or —N(C₁-C₆)-alkyl, with the proviso that heteroatoms are not adjacent;
R², R³ independently of each other are hydrogen, (C₁-C₆)-alkyl, (C₁-C₆)-haloalkyl or halogen, where one or two CH₂ in the alkyl or haloalkyl groups is/are optionally replaced by —O— or —S— or —N(C₁-C₆)-alkyl, with the proviso that heteroatoms are not be adjacent;
R⁴ is hydrogen, (C₁-C₁₀)-alkyl, (C₃-C₁₀)-alkenyl, (C₃-C₁₀)-alkynyl, (C₃-C₁₀)-cycloalkyl, (C₄-C₈)-cycloalkenyl, (C₈-C₁₀)-cycloalkynyl, (C₆-C₁₄)-aryl, (C₃-C₁₀)-heterocyclyl or R⁶, where the radicals mentioned may optionally be are unsubstituted or mono- or polysubstituted;
R⁵ is hydrogen, (C₁-C₁₀)-alkyl, (C₃-C₁₀)-alkenyl, (C₃-C₁₀)-alkynyl, (C₃-C₈)-cycloalkyl, (C₄-C₈)-cycloalkenyl, (C₈-C₁₀)-cycloalkynyl, (C₆-C₁₄)-aryl, (C₃-C₁₀)-heterocyclyl or R⁷, where the radicals mentioned may optionally be are unsubstituted or mono- or polysubstituted;
R⁶, R⁷ independently of each other are —C(W)R⁸, —C(W)OR⁸, —C(W)SR⁸, —C(W)NR⁸₂, —C(W)NR⁸—NR⁸₂, —C(W)NR⁸—NR⁸[C(W)R⁸], —SO₂NR⁸₂, —SO₂OR⁸, —S(O)R⁸, —S(O)₂R⁸, —PWR⁸₂ or —PW(OR⁸)₂;
W is ═O, ═S, ═NOR⁸ or ═NNR⁸₂;
the radicals R⁸ are identical or different and are hydrogen, (C₁-C₆)-alkyl, (C₂-C₆)-alkenyl, (C₂-C₆)-alkynyl, (C₃-C₈)-cycloalkyl, (C₄-C₈)-cycloalkenyl, (C₃-C₈)-cycloalkyl-(C₁-C₄)-alkyl, (C₄-C₈)-cycloalkenyl-(C₁-C₄)-alkyl, (C₃-C₈)-cycloalkyl-(C₂-C₄)-alkenyl, (C₄-C₈)-cycloalkenyl-(C₂-C₄)-alkenyl, (C₁-C₆)-alkyl-(C₃-C₈)-cycloalkyl, (C₂-C₆)-alkenyl-(C₃-C₈)-cycloalkyl, (C₂-C₆)-alkynyl-(C₃-C₈)-cycloalkyl, (C₁-C₆)-alkyl-(C₄-C₈)-cycloalkenyl, (C₂-C₆)-alkenyl-(C₄-C₈)-cycloalkenyl, (C₆-C₁₄)-aryl, or (C₃-C₁₀)-heterocyclyl, where the radicals mentioned are unsubstituted or mono- or polysubstituted; or two radicals R⁸ together optionally form a ring system;
with the proviso that at least one of the radicals R⁴ or R⁵ has one of the meanings defined for R⁶ or R⁷ and that, when R⁵ is —C(═O)R^{a}, where R^{a} is (C₁-C₆)-alkyl or (C₆-C₁₄)-aryl and where the radicals mentioned may optionally be are unsubstituted or mono- or polysubstituted, then R⁴ is hydrogen or optionally unsubstituted or mono- or polysubstituted (C₃-C₁₀)-alkenyl, (C₃-C₁₀)-alkynyl, (C₃-C₁₀)-cycloalkyl, (C₄-C₈)-cycloalkenyl, (C₈-C₁₀)-cycloalkynyl or R⁵.

2. The compound of the formula (I) or a salt thereof as claimed in claim 1 where X is CH.

3. The compound of the formula (I) or a salt thereof as claimed in claim 1 where Y is O.

4. The compound of the formula (I) or a salt thereof as claimed in claim 1 where n is 0.

5. The compound of the formula (I) or a salt thereof as claimed in claim 1 where R¹ is CF₃.

6. The compound of the formula (I) or a salt thereof as claimed in claim 1 where R² and R³ are hydrogen.

7. The compound of the formula (I) or a salt thereof as claimed in claim 1 where R⁴ is hydrogen, (C₁-C₆)-alkyl, or (C₁-C₆)-alkyl which is mono- or polysubstituted by F and/or Cl or R⁶.

8. The compound of the formula (I) or a salt thereof as claimed in claim 1 where R⁵ is (C₁-C₆)-alkyl, (C₃-C₆)-alkenyl, (C₃-C₆)-alkynyl, (C₃-C₈)-cycloalkyl, (C₆-C₁₄)-aryl or (C₃-C₁₀)-heterocyclyl having a total of one to three nitrogen, oxygen and/or sulfur ring atoms or very particularly prefer R⁷, where the radicals mentioned are unsubstituted or mono- or polysubstituted.

9. The compound of the formula (I) or a salt thereof as claimed in claim 1 where R⁶ and R⁷ independently of each other are —C(W)R⁸, —C(W)OR⁸, —SO₂OR⁸, —S(O)R⁸, —S(O)₂R⁸, —PWR⁸₂ or —PW(OR⁸)₂, W is ═O and the radicals R⁸ are identical or different and are (C₁-C₆)-alkyl, (C₂-C₆)-alkenyl, (C₂-C₆)-alkynyl, (C₃-C₈)-cycloalkyl, (C₆-C₁₄)-aryl, or (C₃-C₁₀)-heterocyclyl having a total of one to three nitrogen, oxygen and/or sulfur ring atoms, where the radicals mentioned are unsubstituted or mono- or polysubstituted.

10. The compound of the formula (I) or a salt thereof as claimed in claim 1 where the symbols and indices are as defined below:
X is CH;
Y is O;
n is 0;
R¹ is —CF₃;
R² and R³ are hydrogen;
R⁴ is hydrogen, —C(W)R⁸, —S(O)R⁸ or —S(O)₂R⁸;
R⁵ is (C₁-C₆)-alkyl, (C₃-C₆)-alkenyl, (C₃-C₆)-alkynyl, (C₃-C₈)-cycloalkyl, (C₆-C₁₄)-aryl, (C₃-C₁₀)-heterocyclyl having a total of one to three nitrogen, oxygen and/or sulfur ring atoms, —C(W)R⁸, —S(O)R⁸ or —S(O)₂R⁸; where the radicals mentioned are unsubstituted or mono- or polysubstituted.

11. The compound of the formula (I) or a salt thereof as claimed in claim 10 where the symbols and indices are as defined below:
R⁴ is —C(W)R⁸ or hydrogen;
R⁵ is —C(W)R⁸, and
the radicals R⁸ are identical or different and are hydrogen, (C₁-C₆)-alkyl, (C₂-C₆)-alkenyl, (C₂-C₆)-alkynyl, (C₃-C₈)-cycloalkyl, (C₆-C₁₄)-aryl, or (C₃-C₁₀)-heterocyclyl having a total of one to three nitrogen, oxygen and/or sulfur ring atoms, where the radicals mentioned are unsubstituted or mono- or polysubstituted.

12. The compound of the formula (I) or a salt thereof as claimed in claim 1 where the substituents on the radicals R⁴, R⁵ and R⁸ are groups R⁹ which are as defined below:
the radicals R⁹ are identical or different and are R¹⁰, or two radicals R⁹ together with the atoms to which they are attached form a three- to eight-membered saturated or unsaturated ring system which is optionally substituted by one or more radicals R¹⁰ and which optionally contains heteroatoms O, N, S, SO and/or SO₂;
the radicals R¹⁰ are identical or different and are R⁸, R¹¹, —C(W)R⁸, —C(W)OR⁸, —C(W)SR⁸, —C(W)NR⁸₂, —OC(W)R⁸, —OC(W)OR⁸, —OC(M)SR⁸, —OC(W)NR⁸₂, —SC(W)R⁸, —SC(W)OR⁸, —SC(W)SR⁸, —SC(V)NR⁸₂, —NR⁸C(W)R⁸, —N[C(W)R⁸]₂, —NR⁸C(W)OR⁸, —NR⁸C(W)SR⁸, —C(W)NR⁸—NR⁸₂, —C(W)NR⁸—NR⁸[C(W)R⁸], —NR⁸—C(W)NR⁸₂, —NR⁸—NR⁸C(W)R⁸, —NR⁸—N[C(W)R⁸]₂, —N[(CW)R⁸]—NR⁸₂, —NR⁸[(CW)NR⁸₂], —NR⁸(C═NR⁸)R⁸, —NR⁸(C═NR⁸)NR⁸₂, —O—NR⁸₂, —O—NR⁸(CW)R⁸, —SO₂NR⁸₂, —NR⁸SO₂R⁸, —SO₂OR⁸, —OSO₂R⁸, —OR⁸, —NR⁸₂, —SR⁸, —SiR⁸₃, —PR⁸₂, —P(W)R⁸₂, —SOR⁸, —SO₂R⁸, —PWR⁸₂ or —PW(OR⁸)₂;
or two radicals R¹⁰ together are (W), (═N—R⁸), (═CR₂⁸), (═CHR⁸), or (═CH₂);
W and R⁸ are as defined in claim 1,
the radicals R¹¹ are identical or different and are halogen, cyano, nitro, hydroxyl, thio, amino, formyl, (C₁-C₆)-alkanoyl, (C₁-C₆)-alkoxy, (C₃-C₆)-alkenyloxy, (C₃-C₆)-alkynyloxy, (C₁-C₆)-haloalkyloxy, (C₃-C₆)-haloalkenyloxy, (C₃-C₆)-haloalkynyloxy, (C₃-C₈)-cycloalkoxy, (C₄-C₈)-cycloalkenyloxy, (C₃-C₈)-halocycloalkoxy, (C₄-C₈)-halocycloalkenyloxy, (C₃-C₈)-cycloalkyl-(C₁-C₄)-alkoxy, (C₄-C₈)-cycloalkenyl-(C₁-C₄)-alkoxy, (C₃-C₈)-cycloalkyl-(C₂-C₄)-alkenyloxy, (C₄-C₈)-cycloalkenyl-(C₂-C₄)-alkenyloxy, (C₁-C₆)-alkyl-(C₃-C₈)-cycloalkoxy, (C₂-C₆)-alkenyl-(C₃-C₈)-cycloalkoxy, (C₂-C₆)-alkynyl-(C₃-C₈)-cycloalkoxy, (C₁-C₆)-alkyl-(C₄-C₈)-cycloalkenyloxy, (C₂-C₆)-alkenyl-(C₄-C₈)-cycloalkenyloxy, (C₁-C₄)-alkoxy-(C₁-C₆)-alkoxy, (C₁-C₄)-alkoxy-(C₃-C₆)-alkenyloxy, carbamoyl, (C₁-C₆)-mono- or dialkylcarbamoyl, (C₁-C₆)-mono- or dihaloalkylcarbamoyl, (C₃-C₈)-mono- or dicycloalkylcarbamoyl, (C₁-C₆)-alkoxycarbonyl, (C₃-C₈)-cycloalkoxycarbonyl, (C₁-C₆)-alkanoyloxy, (C₃-C₈)-cycloalkanoyloxy, (C₁-C₆)-haloalkoxycarbonyl, (C₁-C₆)-haloalkanoyloxy, (C₁-C₆)-alkanamido, (C₁-C₆)-haloalkanamido, (C₂-C₆)-alkenam ido, (C₃-C₈)-cycloalkanam ido, (C₃-C₈)-cycloalkyl-(C₁-C₄)-alkanamido, (C₁-C₆)-alkylthio, (C₃-C₆)-alkenylthio, (C₃-C₆)-alkynylthio, (C₁-C₆)-haloalkylthio, (C₃-C₆)-haloalkenylthio, (C₃-C₆)-haloalkynylthio, (C₃-C₈)-cycloalkylthio, (C₄-C₈)-cycloalkenylthio, (C₃-C₈)-halocycloalkthio, (C₄-C₈)-halocycloalkenylthio, (C₃-C₈)-cycloalkyl-(C₁-C₄)-alkylthio, (C₄-C₈)-cycloalkenyl-(C₁-C₄)-alkylthio, (C₃-C₈)-cycloalkyl-(C₃-C₄)-alkenylthio, (C₄-C₈)-cycloalkenyl-(C₃-C₄)-alkenylthio, (C₁-C₆)-alkyl-(C₃-C₈)-cycloalkylthio, (C₂-C₆)-alkenyl-(C₃-C₈)-cycloalkylthio, (C₂-C₆)-alkynyl-(C₃-C₈)-cycloalkylthio, (C₁-C₆)-alkyl-(C₄-C₈)-cycloalkenylthio, (C₂-C₆)-alkenyl-(C₄-C₈)-cycloalkenylthio, (C₁-C₆)-alkylsulfinyl, (C₃-C₆)-alkenylsulfinyl, (C₃-C₆)-alkynylsulfinyl, (C₁-C₆)-haloalkylsulfinyl, (C₃-C₆)-haloalkenylsulfinyl, (C₃-C₆)-haloalkynylsulfinyl, (C₃-C₈)-cycloalkylsulfinyl, (C₄-C₈)-cycloalkenylsulfinyl, (C₃-C₈)-halocycloalkylsulfinyl, (C₄-C₈)-halocycloalkenylsulfinyl, (C₃-C₈)-cycloalkyl-(C₁-C₄)-alkylsulfinyl, (C₄-C₈)-cycloalkenyl-(C₁-C₄)-alkylsulfinyl, (C₃-C₈)-cycloalkyl-(C₃-C₄)-alkenylsulfinyl, (C₄-C₈)-cycloalkenyl-(C₃-C₄)-alkenylsulfinyl, (C₁-C₆)-alkyl-(C₃-C₈)-cycloalkylsulfinyl, (C₂-C₆)-alkenyl-(C₃-C₈)-cycloalkylsulfinyl, (C₂-C₆)-alkynyl-(C₃-C₈)-cycloalkylsulfinyl, (C₁-C₆)-alkyl-(C₄-C₈)-cycloalkenylsulfinyl, (C₂-C₆)-alkenyl-(C₄-C₈)-cycloalkenylsulfinyl, (C₁-C₆)-alkylsulfonyl, (C₃-C₆)-alkenylsulfonyl, (C₃-C₆)-alkynylsulfonyl, (C₁-C₆)-haloalkylsulfonyl, (C₃-C₆)-haloalkenylsulfonyl, (C₃-C₆)-haloalkynylsulfonyl, (C₃-C₈)-cycloalkylsulfonyl, (C₄-C₈)-cycloalkenylsulfonyl, (C₃-C₈)-halocycloalkylsulfonyl, (C₄-C₈)-halocycloalkenylsulfonyl, (C₃-C₈)-cycloalkyl-(C₁-C₄)-alkylsulfonyl, (C₄-C₈)-cycloalkenyl-(C₁-C₄)-alkylsulfonyl, (C₃-C₈)-cycloalkyl-(C₃-C₄)-alkenylsulfonyl, (C₄-C₈)-cycloalkenyl-(C₃-C₄)-alkenylsulfonyl, (C₁-C₆)-alkyl-(C₃-C₈)-cycloalkylsulfonyl, (C₂-C₆)-alkenyl-(C₃-C₈)-cycloalkylsulfonyl, (C₂-C₆)-alkynyl-(C₃-C₈)-cycloalkylsulfonyl, (C₁-C₆)-alkyl-(C₄-C₈)-cycloalkenylsulfonyl, (C₂-C₆)-alkenyl-(C₄-C₈)-cycloalkenylsulfonyl, (C₁-C₆)-dialkylamino, (C₁-C₆)-alkylamino, (C₃-C₆)-alkenylamino, (C₃-C₆)-alkynylamino, (C₂-C₆)-haloalkylamino, (C₃-C₆)-haloalkenylamino, (C₃-C₆)-haloalkynylamino, (C₃-C₈)-cycloalkylamino, (C₄-C₈)-cycloalkenylamino, (C₃-C₈)-halocycloalkamino, (C₄-C₈)-halocycloalkenylamino, (C₃-C₈)-cycloalkyl-(C₁-C₄)-alkylamino, (C₄-C₈)-cycloalkenyl-(C₁-C₄)-alkylamino, (C₃-C₈)-cycloalkyl-(C₃-C₄)-alkenylamino, (C₄-C₈)-cycloalkenyl-(C₃-C₄)-alkenylamino, (C₁-C₆)-alkyl-(C₃-C₈)-cycloalkylamino, (C₂-C₆)-alkenyl-(C₃-C₈)-cycloalkylamino, (C₂-C₆)-alkynyl-(C₃-C₈)-cycloalkylamino, (C₁-C₆)-alkyl-(C₄-C₈)-cycloalkenylamino, (C₂-C₆)-alkenyl-(C₄-C₈)-cycloalkenylamino, (C₁-C₆)-trialkylsilyl, aryl, aryloxy, arylthio, arylamino, aryl-(C₁-C₄)-alkoxy, aryl-(C₃-C₄)-alkenyloxy, aryl-(C₁-C₄)-alkylthio, aryl-(C₂-C₄)-alkenylthio, aryl-(C₁-C₄)-alkylamino, aryl-(C₃-C₄)-alkenylamino, aryl-(C₁-C₆)-dialkylsilyl, diaryl-(C₁-C₆)-alkylsilyl, triarylsilyl and or 5- or 6-membered heterocyclyl, where the cyclic moiety of the 14 last-mentioned radicals is optionally substituted by one or more radicals selected from the group consisting of halogen, cyano, nitro, amino, hydroxyl, thio, (C₁-C₄)-alkyl, (C₁-C₄)-haloalkyl, (C₃-C₈)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylamino, (C₁-C₄)-halo-alkylamino, formyl and (C₁-C₄)-alkanoyl.

13. A process for preparing a compound of the formula (I) as claimed in claim 1, which comprises reacting an activated carboxylic acid derivative of the formula (II) with an hydroxylamine derivatives derivative of the formula (III) 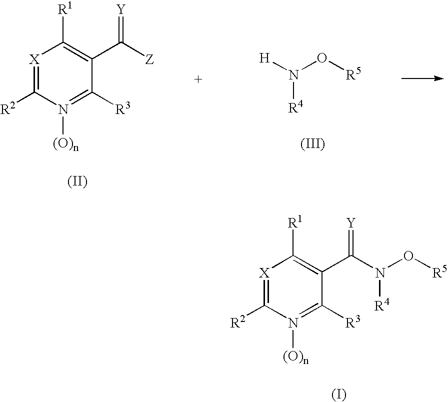
where R¹, R², R³, R⁴, R⁵, X, Y and n are as defined in claim 1 and Z is halogen.

14. A process for preparing compounds of the formula (I) as claimed in claim 1
where R¹, R², R³, R⁴, R⁵, X, Y and n are as defined in claim 1, provided that at least one of the radicals R⁴ or R⁵ is —C(W)R⁸, —C(W)OR⁸, —C(W)SR⁸, —C(W)NR⁸₂, —C(W)NR⁸—NR⁸₂, —C(W)NR⁸—NR⁸[C(W)R⁸], —SO₂NR⁸₂, —SO₂OR⁸, —S(O)R⁸₁—S(O)₂R⁸, —PWR⁸₂ or —PW(OR⁸)₂, which comprises reacting a compound of the formula (I) where R⁴ and R⁵ are hydrogen with a compound of the formula (IV),
R¹²-Hal (IV)
where Hal is a halogen atom and R¹² is a radical selected from the group consisting of —C(W)R⁸, —C(W)OR⁸, —C(W)SR⁸, —C(W)NR⁸₂, —C(W)NR⁸—NR⁸₂, —C(W)NR⁸—NR⁸[C(W) R⁸], —SO₂NR⁸₂, —SO₂OR⁸, —S(O)R⁸, —S(O)₂R⁸, —PWR⁸₂ and —PW(OR⁸)₂, where W and R⁸ are as defined in claim 1.

15. A pesticidal composition, which comprises an insecticidally, acaricidally, ixodicidally, nematicidally or molluscidally effective amount of at least one compound of the formula (I) or a salt thereof as claimed in claim 1 and a suitable formulation auxiliary.

16. The composition as claimed in claim 15, which comprises a further active compound selected from the group consisting of acaricides, fungicides, herbicides, insecticides, nematicides and growth-regulating substances.

17. A method for controlling, deterring or repelling pests including nuisance pests of plants, which comprises treating the plants and/or pests/nuisance pests with a pesticidally effective amount of a compound of formula (I) or a salt thereof as claimed in claim 1.

18. The method as claimed in claim 17 where the plant is a transgenic crop plant.

19. (canceled)

20. A method for controlling endo- and ectoparasites comprising administering to a human or other animal in need of such treatment a Pharmaceutically or veterinarily acceptable, parasiticidally effective amount of a compound of formula (I) or a salt thereof as claimed in claim 1.

21. The compound of the formula (I) or a salt thereof as claimed in claim 1 wherein R⁴ is hydrogen or —C(═O)R⁸ wherein R⁸ is (C₁-C₆)-alkyl, (C₂-C₆)-alkenyl, (C₃-C₈)-cycloalkyl, phenyl or pyridyl, said phenyl or pyridyl being unsubstituted or substituted by one or more radicals selected from the group consisting of halogen, (C₁-C₄)-alkyl, (C₁-C₄)-haloalkyl and (C₁-C₄)-alkoxy.

22. The compound of the formula (I) or a salt thereof as claimed in claim 1 wherein R⁵ is (C₁-C₆)-alkyl or —C(═O)R⁸ wherein R⁸ is (C₁-C₆)-alkyl, (C₂-C₆)-alkenyl, (C₃-C₈)-cycloalkyl, phenyl-(C₁-C₄)-alkoxy or phenyl, phenyl being unsubstituted or substituted by one or more radicals selected from the group consisting of halogen, (C₁-C₄)-alkyl, (C₁-C₄)-haloalkyl and (C₁-C₄)-alkoxy.

23. The compound of the formula (I) or a salt thereof as claimed in claim 1 wherein:
R⁴ is hydrogen or —C(═O)R⁸ wherein R⁸ is (C₁-C₆)-alkyl, (C₂-C₆)-alkenyl, (C₃-C₈)-cycloalkyl, phenyl or pyridyl, each of phenyl and pyridyl being unsubstituted or substituted by one or more radicals selected from the group consisting of halogen, (C₁-C₄)-alkyl, (C₁-C₄)-haloalkyl and (C₁-C₄)-alkoxy;
R⁵ is (C₁-C₆)-alkyl or —C(═O)R⁸ wherein R⁸ is (C₁-C₆)-alkyl, (C₂-C₆)-alkenyl, (C₃-C₈)-cycloalkyl, phenyl-(C₁-C₄)-alkoxy or phenyl, phenyl being unsubstituted or substituted by one or more radicals selected from the group consisting of halogen, (C₁-C₄)-alkyl, (C₁-C₄)-haloalkyl and (C₁-C₄)-alkoxy.
with the proviso that at least one of R⁴ and R⁵ is —C(═O)R⁸.

24. The compound or salt as claimed in claim 1, having the formula 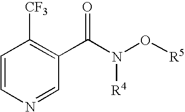
wherein:
(a) R⁴ is hydrogen and R⁵ is —CO—CH(CH₃)₂;
(b) R⁴ is —CO-(4-trifluoromethylpyridyl-3-yl) and R⁵ is —CH₃;
(c) R⁴ is —CO-(4-trifluoromethylpyridyl-3-yl) and R⁵ is —C₂H₅;
(d) R⁴ is hydrogen and R⁵ is —CO—CH₂—O—C₆H₅;
(e) R⁴ is —CO-(2-methoxyphenyl) and R⁵ is —CO-(2-methoxyphenyl);
(f) R⁴ is hydrogen and R⁵ is —CO-(2-chloro-4-trifluorophenyl);
(g) R⁴ is —CO-(2-chlorophenyl) and R⁵ is —CO-(2-chlorophenyl);
(h) R⁴ is hydrogen and R⁵ is —CO-(2-chlorophenyl);
(i) R⁴ is hydrogen and R⁵ is —CO-tert-butyl;
(j) R⁴ is —CO-cyclopropyl and R⁵ is —CO-cyclopropyl;
(k) R⁴ is —CO—CH═CH—CH₃ and R⁵ is —CO—CH═CH—CH₃;
(l) R⁴ is hydrogen and R⁵ is —CO-(4-methylphenyl);
(m) R⁴ is —CO-phenyl and R⁵ is —CO-phenyl;
(n) R⁴ is —CO—CH₃ and R⁵ is —CO—CH₃; or
(o) R⁴ is hydrogen and R⁵ is —CO—CH₃.

25. The compound or salt as claimed in claim 1, having the formula 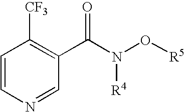
wherein:
(a) R⁴ is hydrogen and R⁵ is —CO—CH(CH₃)₂;
(b) R⁴ is —CO-(4-trifluoromethylpyridyl-3-yl) and R⁵ is —CH₃;
(c) R⁴ is —CO-(2-methoxyphenyl) and R⁵ is —CO-(2-methoxyphenyl);
(d) R⁴ is hydrogen and R⁵ is —CO-(2-chloro-4-trifluorophenyl);
(e) R⁴ is —CO-(2-chlorophenyl) and R⁵ is —CO-(2-chlorophenyl);
(f) R⁴ is hydrogen and R⁵ is —CO-(2-chlorophenyl);
(g) R⁴ is hydrogen and R⁵ is —CO-tert-butyl;
(h) R⁴ is —CO-cyclopropyl and R⁵ is —CO-cyclopropyl;
(i) R⁴ is —CO—CH═CH—CH₃ and R⁵ is —CO—CH═CH—CH₃;
(j) R⁴ is —CO-phenyl and R⁵ is —CO-phenyl;
(k) R⁴ is —CO—CH₃ and R⁵ is —CO—CH₃; or
(l) R⁴ is hydrogen and R⁵ is —CO—CH₃.
